(19) United States
(12) Patent Application Publication
(10) Pub. No.: US 2014/0045751 A1
(43) Pub. Date: Feb. 13, 2014

(54) RECOMBINANT HUMAN FIBROBLAST GROWTH FACTOR-1 AS A NOVEL THERAPEUTIC FOR ISCHEMIC DISEASES AND METHODS THEREOF

(71) Applicant: FLORIDA STATE UNIVERSITY RESEARCH FOUNDATION, Tallahassee, FL (US)

(72) Inventor: Michael BLABER, Tallahassee, FL (US)

(73) Assignee: FLORIDA STATE UNIVERSITY RESEARCH FOUNDATION, Tallahassee, FL (US)

(21) Appl. No.: 13/963,023

(22) Filed: Aug. 9, 2013

Related U.S. Application Data
(60) Provisional application No. 61/681,819, filed on Aug. 10, 2012.

(51) Int. Cl. C07K 14/50

(52) U.S. Cl. CPC C07K 14/50
USPC 514/9.1

(57) ABSTRACT

Described is method, comprising the following step: administering to an individual having an ischemic condition or disease a composition comprising a mutant fibroblast growth factor (FGF) protein having a polypeptide sequence that is at least 90% identical to the polypeptide sequence of wild-type human FGF-1 protein, wherein the numbering of the amino acid positions is based on the numbering scheme for the 140 amino acid form of human FGF-1.

### CROSS-REFERENCE TO RELATED APPLICATIONS

This application claims benefit of priority to U.S. Provisional Patent Application No. 61/681,819, entitled “PHARMACOKINETIC PROPERTIES OF 2^{ND}-GENERATION FIBROBLAST GROWTH FACTOR-1 MUTANTS FOR THERAPEUTIC APPLICATION”, filed Aug. 10, 2012 which is incorporated herein by reference in its entirety.

### BACKGROUND

1. Field of the Invention

The present invention relates to recombinant and engineered fibroblast growth factor-1 for the treatment of ischemic diseases.

2. Related Art

FGF-1 has low intrinsic thermostability and is characteristically formulated with heparin as a stabilizing agent. Heparin, however, adds a number of undesirable properties that negatively impact safety and cost.

### SUMMARY

According to a first broad aspect, the present invention provides a method comprising the following step: (a) administering to an individual having an ischemic condition or disease a composition comprising a mutant fibroblast growth factor (FGF) protein having a polypeptide sequence that is at least 90% identical to the polypeptide sequence of wild-type human FGF-1 protein (SEQ ID NO: 2), wherein the numbering of the amino acid positions is based on the numbering scheme for the 140 amino acid form of human FGF-1, and wherein the composition is administered without heparin.

According to a second broad aspect, the present invention provides a method comprising the following step: (a) administering to an individual having an ischemic condition or disease a composition comprising a mutant fibroblast growth factor (FGF) protein having a polypeptide sequence that is at least 90% identical to the polypeptide sequence of wild-type human FGF-1 protein (SEQ ID NO: 3), wherein the numbering of the amino acid positions is based on the numbering scheme for the 140 amino acid form of human FGF-1, and wherein the composition is administered without heparin.

According to a second broad aspect, the present invention provides a method comprising the following step: (a) administering to an individual having an ischemic condition or disease a composition comprising a mutant fibroblast growth factor (FGF) protein having a polypeptide sequence that is at least 90% identical to the polypeptide sequence of wild-type human FGF-1 protein (SEQ ID NO: 4), wherein the numbering of the amino acid positions is based on the numbering scheme for the 140 amino acid form of human FGF-1, and wherein the composition is administered without heparin.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a diagram showing the relationship between wild-type FGF-1 and mutant proteins.

FIG. 2 is a table listing the proteins utilized in the pharmokinetic study in this present invention.

FIG. 3 is a table showing a two-compartment IV bolus pharmacokinetic model (Cp=A×e^{-αt}+B×e^{-βt}) analysis of wild-type and mutant FGF-1 proteins (n=3 for each protein).

FIG. 4 is a graph showing plasma glucose levels for the 0-1440 min time period after IV bolus of PBX (control) buffer.

FIG. 5 is a graph showing plasma glucose levels for the 0-1440 min time period after IV bolus of wild-type FGF-1 plus heparin.

FIG. 6 is a graph showing plasma glucose levels for the 0-1440 min time period after IV bolus of wild-type FGF-1 without heparin.

FIG. 7 is a graph showing plasma glucose levels for the 0-1440 min time period after IV bolus of mutant M1 (SEQ ID NO: 2).

FIG. 8 is a graph showing plasma glucose levels for the 0-1440 min time period after IV bolus of mutant M2 (SEQ ID NO: 3).

FIG. 9 is a graph showing plasma glucose levels for the 0-1440 min time period after IV bolus of mutant M3 [SEQ ID NO: 4].

FIG. 10 is a graph showing pharmacokinetic mean residence time (MRT) values for all proteins.

FIG. 11 is a graph showing pharmacokinetic Cp*t curves for all proteins.

FIG. 12 is a diagram showing representative silver stained SDS-PAGE of purified recombinant protein (4 μg FGF-1) resolved in the presence and absence of DTT reducing agent.

FIG. 13 is graph showing representative Log(Cp) vs. time data (averaged FGF without heparin data set; n=3) and two-compartment model fit.

FIG. 14 is a table showing the plasma concentration (Cp) of FGF-1 and mutant proteins and time points utilized in the PK analysis. The values and standard deviations are for n=3 in each case.

FIG. 15 is a graph showing the PK profile and fitted two-compartment model for wild-type and mutant FGF-1 proteins (error bars for n=3 data sets for each protein).

FIG. 16 is a graph showing plasma triglyceride levels for pre-bleed (T=0) control, 240, 480 and 1440 min samples for FGF-1 and mutant proteins after IV bolus.

FIG. 17 is a table showing values of plasma triglyceride (mg/dL) and cholesterol levels (mg/dL).

FIG. 18 is a graph showing plasma cholesterol levels for pre-bleed (T=0) control, 240, 480 and 1440 min samples.

FIG. 19 is a graph showing plasma alanine transaminase levels from a liver panel analysis for the 1440 min time point after IV bolus of PBX buffer control, FGF-1 w/o heparin, or mutant M2 protein.

FIG. 20 is a graph showing plasma aspartate transaminase levels from a liver panel analysis for the 1440 min time point after IV bolus of PBX buffer control, FGF-1 w/o heparin, or mutant M2 protein.

FIG. 21 is a graph showing plasma bilirubin levels from a liver panel analysis for the 1440 min time point after IV bolus of PBX buffer control, FGF-1 w/o heparin, or mutant M2 protein.

FIG. 22 is a graph showing plasma albumin levels from a liver panel analysis for the 1440 min time point after IV bolus of PBX buffer control, FGF-1 w/o heparin, or mutant M2 protein FIG. 23 is a graph showing plasma γ-glutamyltranspeptidase (GGT) levels from a liver panel analysis for the 1440 min time point after IV bolus of PBX buffer control, FGF-1 w/o heparin, or mutant M2 protein.

FIG. 24 is a table showing values of 24 hour time point liver chemistry profiles that correspond with those shown in FIGS. 19, 20, 21, 22 and 23.

FIG. 25 is a table showing values of plasma glucose levels (mg/dL) that correspond with those shown in FIGS. 4, 5, 6, 7, 8 and 9.

### DETAILED DESCRIPTION OF THE PREFERRED EMBODIMENTS

### Definitions

Where the definition of terms departs from the commonly used meaning of the term, applicant intends to utilize the definitions provided below, unless specifically indicated.

For purposes of the present invention, it should be noted that the singular forms, “a,” “an” and “the” include reference to the plural unless the context as herein presented clearly indicates otherwise.

For purposes of the present invention, the term “amino acid” refers to a biological organic compound that is coded for by a genetic code of an organism and is a precursor to protein.

For purposes of the present invention, the term “angiogenesis” and the term “angiogenetic” refer to a physiological process through which new blood vessels form from pre-existing vessels.

For purposes of the present invention, the term “correspond” and the term “corresponding” in reference to a protein sequence refer interchangeably to an amino acid position(s) of a protein, such as a mutant FGF protein, that is equivalent or corresponds to an amino acid position(s) of one or more other protein(s), such as a wild-type FGF protein, according to any standard criteria known in the art. An amino acid at a position of a protein may be found to be equivalent or corresponding to an amino acid at a position of one or more other protein(s) based on any relevant evidence, such as the primary sequence context of the each amino acid, its position in relation to the N-terminal and C-terminal ends of its respective protein, the structural and functional roles of each amino acid in its respective protein, etc. For proteins having similar or nearly identical polypeptide sequences, a “corresponding” amino acid(s) and corresponding amino acid position(s) between proteins may be determined or deduced by sequence alignment and comparison. However, “corresponding” amino acid(s) for two or more proteins may have different amino acid position numbers or numbering (e.g., when counted from the N-terminus of each protein) since the two or more proteins may have different lengths and/or one or more substitutions, insertions, deletions, etc. For example, related proteins may have deletions or insertions in relation to each other that offset the numbering of their respective or corresponding amino acid sequences (i.e., based on their primary structure or sequence). An amino acid position(s) of a protein, such as a mutant FGF protein, may “correspond” to an amino acid position(s) of one or more other protein(s) if the amino acid positions are structurally equivalent or similar when comparing the three-dimensional structures (i.e., tertiary structures) of the respective proteins. A person skilled in the art would be able to determine “corresponding” amino acids and/or “corresponding” amino acid positions of two or more proteins based on their protein sequences and/or protein folding or tertiary structure.

For purposes of the present invention, the term “deletion” refers to the absence of an amino acid residue from the polypeptide sequence of a mutant protein.

For purposes of the present invention, the term “functional half-life” of a FGF protein refers to the amount of time it takes for the activity or effect of a FGF protein (e.g., a mutant FGF protein) to become reduced by half. For example, the functional half-life may be based on the activity of a FGF protein over time in inducing growth, proliferation, and/or survival of cells, such as according to a cultured fibroblast proliferation assay. The functional half-life of a protein may be different than the thermostability of the same protein since these are separable properties of proteins. For example, a protein may be mutated such that the thermostability of the protein is decreased while its functional half-life is increased.

For purposes of the present invention, the term “growth factor” refers to a naturally occurring substance capable of stimulating cellular growth, proliferation and cellular differentiation. It is usually a protein or a steroid hormone and is important for regulating a variety of cellular processes.

For purposes of the present invention, the terms “identical” or “identity” refer to the percentage of amino acid residues of two or more polypeptide sequences having the same amino acid at corresponding positions. For example, a protein that is at least 90% identical to a polypeptide sequence will have at least 90% of its residues that are the same as those in the polypeptide sequence at corresponding positions.

For purposes of the present invention, the terms “individual,” “subject,” or “patient” refer interchangeably to a mammalian organism, such as a human, mouse, etc., that is administered a mutant FGF protein of the present invention for a therapeutic or experimental purpose.

For purposes of the present invention, the term “mutation” refers to a change in the polypeptide sequence of a protein.

For purposes of the present invention, the term “polypeptide” refers to a polymer chain of amino acids joined together by peptide that is unbranched.

For purposes of the present invention, the term “substitution” refers to the replacement of an amino acid residue at a specific position along the polypeptide sequence of a mutant protein.

For purposes of the present invention, the terms “thermodynamic stability” or “thermostability” of a protein refer interchangeably to the ability of a protein to maintain its tertiary structure (i.e., to resist denaturation or unfolding) at a given temperature or in the presence of a denaturant. The thermostability of a protein (e.g., a mutant FGF protein) may be expressed in terms of the Gibb's free energy equation relative to a standard (e.g., wild-type FGF protein) according to known methods.

For purposes of the present invention, the term “thiol” refers to an organosulfur compound that contains a carbon-bonded sulfhydryl (—C—SH or R—SH) group where R represents an alkane, alkene, or other carbon-containing group of atoms.

### DESCRIPTION

Protein biopharmaceuticals are an important and growing area of human therapeutics, but the intrinsic property of proteins to adopt alternative conformations (e.g., during protein unfolding) presents numerous challenges limiting their effective application as biopharmaceuticals. Although still a comparatively small percentage overall, protein biopharmaceuticals are the fastest-growing category of new drug approvals and currently target over 200 human diseases, including cancers, heart disease, Alzheimer's, diabetes, multiple sclerosis, AIDS, and arthritis. See, e.g., Crommelin, D. J. A., et al., “Shifting paradigms: biopharmaceuticals versus low molecular weight drugs,” International Journal of Pharmaceutics 266:3-16 (2003), the entire contents and disclosure of which are hereby incorporated by reference. The impact of protein biopharmaceuticals upon U.S. healthcare and the economy is substantial and growing rapidly. However, in comparison to traditional small molecules, proteins present new and significant challenges that need to be overcome before their full potential as therapeutic agents may be realized. One unique property of proteins is that they are able to adopt different structural conformations, and this profoundly influences critically-important properties of proteins, such as their function, solubility, bioavailability, half-life, aggregation, toxicity, immunogenicity, etc. See, e.g., Frokjaer, S. et al., “Protein drug stability: a formulation challenge,” Nature Reviews 4:298-306 (2005); Hermeling, S. et al., “Structure-immunogenicity relationships of therapeutic proteins,” Pharmaceutical Research 21:897-903 (2004); and Krishnamurthy, R. et al., “The stability factor: importance in formulation development,” Current Pharmaceutical Biotechnology 3:361-371 (2002), the entire contents and disclosures of which are hereby incorporated by reference. A key intrinsic property of proteins in this regard is their thermodynamic stability (ΔG_{unfolding}) which defines an equilibrium between native and denatured states of a protein.

The thermodynamic stability of a protein may be of particular significance in therapeutic applications because unfolded or aggregated forms of a protein may be potentially toxic and/or immunogenic. For example, neutralizing antibodies in patients treated with interferon-alpha 2a were observed when the protein is stored at room temperature and formed detectable aggregates, but both the formation of aggregates and immunogenicity were reduced upon storage at 4° C. (where ΔG_{unfolding} increased). See, e.g., Hochuli, E. “Interferon immunogenicity: technical evaluation of interferon-alpha 2a,” Journal of Interferon and Cytokine Research 17:S15-S21 (1997), the entire contents and disclosure of which are hereby incorporated by reference. In a different study, persistent antibodies were generated in patients treated with human growth hormone with formulations containing 50-70% aggregates. However, when the formulation of human growth hormone was modified to contain less than 5% aggregates, only transient or no antibodies were observed. See, e.g., Moore, W. V. et al., “Role of aggregated human growth hormone (hGH) in development of antibodies to hGH,” Journal of Clinical Endocrinology and Metabolism 51:691-697 (1980), the entire contents and disclosure of which are hereby incorporated by reference. In yet another study using recombinant clotting factor VIII in mice, the formation of aggregates was associated with the emergence of entirely novel immunogenic epitopes. See, e.g., Purohit, V. S. et al., “Influence of aggregation on immunogenicity of recombinant human Factor VIII in hemophilia A mice,” Journal of Pharmaceutical Sciences 95:358-371 (2006), the entire contents and disclosure of which are hereby incorporated by reference. Thus, protein stability, denaturation, aggregation and immunogenicity may be critical and interrelated issues influencing the successful application of proteins as biopharmaceuticals.

Various efforts have been made to increase the thermodynamic stability and/or half-life of proteins that are intended for use as biopharmaceuticals, while reducing their aggregation and/or immunogenicity. One such approach uses covalent attachment of polyethylene glycol (PEG), a highly soluble and biocompatible polymer, to substantially increase the circulating half-life of proteins through reduced renal clearance due to a substantial increase in the molecular mass of these proteins. The attached PEG molecule may also physically mask regions of the protein that would otherwise be susceptible to proteolytic attack or immune recognition, increasing further the circulating half-life and reducing immunogenicity. However, attachment of PEG molecules (“PEGylation”) typically does not increase the formal thermodynamic stability of proteins and has been noted to reduce the thermodynamic stability in some cases. See, e.g., Basu, A., et al., “Structure-function engineering of interferon-β-1b for improving stability, solubility, potency, immunogenicity, and pharmacokinetic properties by site-selective mono-PEGylation,” Bioconjugate Chemistry 17 (2006); and Monfardini, C. et al., “A branched monomethoxypoly(ethylene glycol) for protein modification,” Bioconjugate Chemistry 6:62-69 (1995), the entire contents and disclosure of which are hereby incorporated by reference.

Therefore, the beneficial properties of PEGylation are primarily associated with modulation of renal clearance and a reduction in proteolysis and immune recognition (i.e., PEGylation generally does not increase thermodynamic stability of a protein). One problem with PEGylation is that it may interfere with critical functional interfaces on the protein surface, often reducing receptor/ligand affinity by two or more orders of magnitude. However, PEGylation studies do show that shielding of epitopes on the protein surface may substantially reduce or eliminate the immunogenic potential of a protein, which may have important ramifications for protein engineering by suggesting that mutations at solvent-inaccessible positions within proteins may minimize their immunogenic potential.

The fibroblast growth factors (gene=Fgf, protein=FGF) are a family of polypeptides with diverse roles in development and metabolism. Fgfs have been found in many multicellular organisms, ranging from Caenorhabditis elegans to Homo sapiens. Two Fgf genes have been identified in C. elegans, while the mouse and human each share 22 Fgf genes. See, e.g., Itoh, N. et al., “Evolution of the Fgf and Fgfr gene families,” Trends Genet 20 (2004); Popovici, C. et al., “An evolutionary history of the FGF superfamily,” BioEssays 27:849-857 (2005); and Itoh, N. et al., “Functional evolutionary history of the mouse Fgf gene family,” Developmental Dynamics 237:18-27 (2008), the entire contents and disclosures of which are hereby incorporated by reference. Fgf genes generally encode potent mitogens for a broad spectrum of cell types, including vascular cells.

Most FGFs mediate their biological responses as extracellular proteins by binding to and activating cell surface tyrosine kinase FGF receptors (FGFRs). Four Fgfr genes, Fgfr1 through Fgfr4, have been identified in humans and mice, and these may be alternatively spliced to produce a greater number of FGFR isoforms. Except for FGF-11 through FGF-14, FGF-15/19, FGF-21, and FGF-23, other FGFs activate FGFRs with high affinity and with different degrees of specificity. See, e.g., Itoh, N. et al. (2008), supra. FGF-1 is the only known wild-type mouse/human FGF protein that is believed to bind to all FGFR types, but other FGF proteins have been shown to bind to multiple FGFRs. See, e.g., Zhang, X. et al., “Receptor Specificity of the Fibroblast Growth Factor Family,” J Biol Chem 281(23):15694-15700 (2006); and Szlachcic et al. (2009), supra, the entire contents and disclosure of which are hereby incorporated by reference. Therefore, a mutant FGF protein according to embodiments of the present invention may bind with specificity and affinity to at least one FGFR present on the surface of a cell, such as a fibroblast cell, neuronal or neuroblast cell, endothelial cell, chondrocyte, osteoblast, myoblast, smooth muscle cell, or glial cell. A mutant FGF protein according to embodiments of the present invention may also trigger growth, proliferation, and/or survival of cells, such as fibroblast cells, neuronal or neuroblast cells, endothelial cells, chondrocytes, osteoblasts, myoblasts, smooth muscle cells, glial cells, etc., known to express one or more FGFRs. This may occur through binding and activation of a FGF receptor and downstream signaling within the cell.

Human and mouse Fgf genes and proteins may be divided into seven subfamilies based on phylogenetic analysis: Fgf-1 or FGF A subfamily (including FGF-1 and FGF-2 proteins); Fgf-4 or FGF C subfamily (including FGF-4, FGF-5, and FGF-6 proteins); Fgf-7 or FGF B subfamily (including FGF-3, FGF-7, FGF-10, and FGF-22 proteins); Fgf-8 or FGF D subfamily (including FGF-8, FGF-17, and FGF-18 proteins); Fgf-9 or FGF E subfamily (including FGF-9, FGF-16, and FGF-20 proteins); intracellular iFgf or FGF F subfamily (including FGF-11, FGF-12, FGF-13, and FGF-14 proteins); and hormone-like hFgf or FGF G subfamily (including FGF-15/FGF-19, FGF-21, and FGF-23 proteins). See, e.g., Itoh, N. et al. (2008), supra; Popovici, C. et al. (2005), supra; and Ornitz, D. M. et al., “Fibroblast growth factors,” Genome Biology 2(3):3005.1-3005.12 (2001), the entire contents and disclosure of which are hereby incorporated by reference.

Several members of the FGF family of proteins, including Fgf-1 subfamily FGF-1 and FGF-2 proteins (also referred to as acidic FGF and basic FGF, respectively), have the potential of providing “angiogenic therapy” for the treatment of ischemic conditions or diseases (i.e., diseases caused by insufficient blood flow to one or more tissues), such as coronary artery disease, peripheral vascular disease, peripheral arterial occlusion or disease (e.g., critical limb ischemia or CLI), etc., by triggering neovascularization of affected tissues. See, e.g., Nikol, S. et al., “Therapeutic Angiogenesis With Intramuscular NV1FGF Improves Amputation-free Survival in Patients With Critical Limb Ischemia,” Mol Ther 16(5):972-978 (2008), the entire contents and disclosure of which are hereby incorporated by reference. In addition, FGF proteins may be used for tissue repair and wound healing by triggering angiogenesis and proliferation of fibroblasts involved in healing damaged tissue and filling the wound space with new tissue.

FGF-1 has also been suggested for use in regenerating nervous system tissue following spinal cord injury or trauma, such as brachial plexus injury, neuroimmunologic disorders, such as acute or idiopathic transverse myelitis (TM), or any other disease or condition where regeneration and/or protection of neurons or neural tissue is desired, since FGF-1 is believed to stimulate neural proliferation and growth and may be neuroprotective. See, e.g., Lin P-S. et al., “Spinal Cord Implantation with Acidic Fibroblast Growth Factor as a Treatment for Root Avulsion in Obstetric Brachial Plexus Palsy,” J Chin Med Assoc 68(8):392-396 (2005); Cheng, H. et al., “Spinal Cord Repair with Acidic Fibroblast Growth Factor as a Treatment for a Patient with Chronic Paraplegia,” SPINE 29(14):E284-E288 (2004); and Lin, P-H., “Functional recovery of chronic complete idiopathic transverse myelitis after administration of neurotrophic factors,” Spinal Cord 44:254-257 (2006), the entire contents and disclosures of which are hereby incorporated by reference.

Pharmaceutical or therapeutic administration of FGF proteins, such as FGF-1 and FGF-2, is limited by the fact that wild-type FGF proteins have poor thermodynamic stability and a short half-life. See, e.g., Szlachcic, A. et al., “Structure of a highly stable mutant of human fibroblast growth factor 1,” Acta Cryst. D65:67-73 (2009), the entire contents and disclosures of which are hereby incorporated by reference. For example, FGF-1 has poor thermodynamic stability with a melting temperature (i.e., a midpoint of thermal denaturation, or Tₘ) that is only marginally above physiological temperature. See, e.g., Copeland, R. A., et al., “The structure of human acidic fibroblast growth factor and its interaction with heparin,” Archives of Biochemistry and Biophysics 289:53-61 (1991), the entire contents and disclosure of which are hereby incorporated by reference. The functional half-life of wild-type FGF-1 in unconditioned DMEM is only about 1.0 hour according to a cultured fibroblast proliferation assay. Although incubation experiments in TBS buffer demonstrate aggregation and loss of soluble monomeric of FGF-1 over a different time scale, these studies do show that loss of soluble monomeric FGF-1 protein over time is due to irreversible aggregation with soluble FGF-1 protein showing formation of higher-mass disulfide adducts. Because of its intrinsic property of instability, FGF-1 is prone to both aggregation and proteolysis, which may cause immunogenicity. Accordingly, substantial effort has been spent on identifying appropriate formulations to counteract these intrinsic properties often with mixed success.

FGF-1 is a “heparin-binding” growth factor, and upon binding of heparin, the Tₘ, of FGF-1 increases by about 20° C., and heparin-bound FGF-1 exhibits reduced susceptibility to denaturation-induced aggregation, thiol reactivity, and proteolytic degradation. See, e.g., Copeland R. A. et al. (1991), supra; and Gospodarowicz, D. et al., “Heparin protects basic and acidic FGF from inactivation,” Journal of Cellular Physiology 128:475-484 (1986), the entire contents and disclosures of which are hereby incorporated by reference. Therefore, one approach to overcoming the therapeutic limitation of FGF protein instability is to administer FGF-1 bound to heparin. Indeed, FGF-1 formulated with the addition of heparin as a protein biopharmaceutical is currently in phase II clinical trials (NCT00117936) for pro-angiogenic therapy in coronary heart disease. However, heparin adds considerable additional expense, has its own pharmacological properties (e.g., it is an anti-coagulant), is derived from animal tissues (with associated concerns regarding the potential for infectious agents), and causes adverse inflammatory or allergic reactions in a segment of the population. Thus, formulation efforts to modulate the physical properties of a protein are often difficult to achieve and can introduce undesired additional cost or side effects.

An alternative approach to “PEGylation” or formulation with heparin is to alter the physical properties of an FGF protein by mutagenesis. By changing its amino acid sequence, an FGF protein may have greater thermodynamic stability and/or increased functional half-life as well as increased solubility and resistance to proteolytic degradation, aggregation, or immunogenic potential. Mutating proteins to improve their properties for human therapeutic application is a viable approach. For example, over thirty mutant forms of proteins have been approved by the FDA for use as human biopharmaceuticals. See, e.g., Kurtzman, A. L. et al., “Advances in directed protein evolution by recursive genetic recombination: applications to therapeutic proteins,” Curr Opin Biotech 12:361-370 (2001), the entire contents and disclosures of which are hereby incorporated by reference. Examples include mutations that contribute to increased yields during purification, increased in vivo functional half-life, or increased specific activity, such as mutations of buried free-cysteine residues in β-interferon (Betaseron®) and interleukin-2 (Proleukin®) as well as others hypothesized to increase thermostability. Thus, a mutational approach to improve the physical properties of proteins is a viable route to develop “second-generation” protein biopharmaceuticals having improved thermodynamic stability and/or functional half-life.

However, the concepts of thermodynamic stability and half-life are separable properties of a protein. For example, as described further below, mutation of buried free cysteine residues of proteins may increase their functional half-life despite causing a reduced thermodynamic stability because mutation of these reactive free cysteine residues avoids the irreversible formation of disulfide bonds that are incompatible with the native conformation of the protein. Therefore, combining separate mutations that eliminate free cysteines with other mutations that increase the thermostability of a protein may have a synergistic effect on the half-life of the protein by avoiding the irreversible denaturation pathway resulting from thiol reactivity of the free cysteine (e.g., disulfide formation) while simultaneously increasing the thermodynamic stability of the protein. This is especially true considering that protein unfolding, which is dependent on protein stability, is often a necessary first step for the irreversible denaturation pathway resulting from exposure of the reactive free cysteine.

The fundamental FGF protein structure is described by a ˜120 amino acid domain that forms a β-trefoil architecture. See, e.g., Murzin, A. G. et al., “β-Trefoil fold. Patterns of structure and sequence in the kunitz inhibitors interleukins-1β and 1α and fibroblast growth factors,” Journal of Molecular Biology 223:531-543 (1992), the entire contents and disclosure of which are hereby incorporated by reference. Among the 22 members of the mouse/human FGF family, three positions are absolutely conserved, and include Gly71, Cys83, and Phe132 (using numbering scheme of the 140 amino acid form of FGF-1). Gly71 is located at the i+3 position in a type 1 β-turn and is the statistically-preferred residue at this position due to structural considerations of backbone strain. See, e.g., Hutchinson, E. G. et al., “A revised set of potentials for beta-turn formation in proteins,” Protein Sci 3:2207-16 (1994); Guruprasad, K. et al., “Beta- and gamma-turns in proteins revisited: a new set of amino acid turn-type dependent positional preferences and potentials,” J Biosci 25:143-56 (2000); Kim, J. et al., “Identification of a key structural element for protein folding within β-hairpin turns,” Journal of Molecular Biology 328:951-961 (2003); and Lee, J. et al., “A logical OR redundancy with the Asx-Pro-Asx-Gly type I β-turn motif,” Journal of Molecular Biology 377:1251-1264 (2008), the entire contents and disclosures of which are hereby incorporated by reference.

Fibroblast growth factor-1 (FGF-1) is an angiogenic factor with therapeutic potential for the treatment of ischemic disease. FGF-1 has low intrinsic thermostability and is characteristically formulated with heparin as a stabilizing agent. Heparin, however, adds a number of undesirable properties that negatively impact safety and cost. Mutations that increase the thermostability of FGF-1 may obviate the need for heparin in formulation and may prove to be useful “2nd-generation” forms for therapeutic use. A pharmacokinetic (PK) study in rabbits of human FGF-1 in the presence and absence of heparin has been reported, as well as three mutant forms having differential effects upon thermostability, buried reactive thiols, and heparin affinity. The results support the hypothesis that heparan sulfate proteoglycan (HSPG) in the vasculature of liver, kidney and spleen serves as the principle peripheral compartment in the distribution kinetics. The addition of heparin to FGF-1 is shown to increase endocrine-like properties of distribution. Mutant forms of FGF-1 that enhance thermostability or eliminate buried reactive thiols demonstrate a shorter distribution half-life, a longer elimination half-life, and a longer mean residence time (MRT) in comparison to wild-type FGF-1 [SEQ ID NO: 1]. The results show how such mutations can produce useful 2nd-generation forms with tailored PK profiles for specific therapeutic application.

A number of major diseases have an insufficiency in blood flow (ischemia) as a primary contributing pathology. Coronary ischemia, peripheral artery disease, and chronic non-healing wounds in diabetic ulcers and bed sores, for example, are fundamentally ischemic diseases. Specific cell types are associated with the growth of new blood vessels and wound healing, including endothelial cells, fibroblasts, and keratinocytes. Almost 50 years ago, normal human proteins capable of causing the proliferation of specific cell types were identified and termed “growth factors” [1, 2, 3, 4]. More recent pre-clinical and clinical studies have demonstrated beneficial effects of the application of such growth factors in the treatment of ischemic disease; studies have reported the effective growth of new blood vessels in cardiac muscle of “no-option” heart patients after injection of a pro-angiogenic growth factor at the site of ischemia [5, 6, 7], as well as substantial acceleration of wound healing with topical application of pro-angiogenic growth factors at the site of full-thickness dermal injury and diabetic ulcers [8, 9, 10, 11, 12].

Several different growth factors have been evaluated in such “pro-angiogenic therapy” including vascular endothelial cell growth factor (VEGF) [13, 14, 15], FGF-1 [7, 9, 10, 16], FGF-2 [17, 18], platelet derived growth factor (PDGF) [19, 20] and keratinocyte growth factor (KGF) [21, 22]. Promising clinical results have been achieved, for example, using fibroblast growth factor-1 (FGF-1): FGF-1 is currently in NIH phase II clinical trials for the pro-angiogenic treatment of coronary heart disease. (NCT00117936) and wound healing in diabetic foot ulcers (NCT00916292). FGF-1 is a relatively simple protein and can be expressed recombinantly using inexpensive bacterial fermentation. However, there are several issues that complicate the practical application of FGF-1 as a therapeutic agent. FGF-1 has an intrinsically poor thermostability (i.e., DGunfolding=21.1 kJ/mol [23]) and is prone to unfolding, aggregation, and subsequent loss of functional activity. Formulation studies to address the poor thermostability of FGF-1 have resulted in the common addition of heparin, which binds to, and stabilizes, FGF-1 [24, 25]. However, heparin is more expensive than FGF-1 to produce, is derived from pigs (with the potential for infectious agents), is highly heterogeneous, has its own (anti-coagulant) pharmacological properties, can initiate a serious allergic reaction, and can cause thrombocytopenia [26, 27]. Furthermore, while the treatment of coronary ischemia by FGF-1 has shown effectiveness with a single (injected) dose, healing of wounds is best achieved with multiple (topical) dosing—necessitating frequent removal of bandages for access to the wound area [8, 9, 10, 28]. This latter issue is complicated by the presence of heparin, which, due to its pharmacological properties, causes wounds to produce excessive exudate, diluting topically-applied therapeutics.

A potentially elegant solution to address negative aspects associated with heparin as a formulation additive for FGF-1 is to engineer the protein to increase the intrinsic thermostability. Such designed mutant proteins represent novel 2nd-generation forms. Over thirty different types of 2nd-generation recombinant human proteins with enhanced properties have been approved by the FDA for use as human pharmaceuticals [29]; examples include binterferon (BetaseronH) and interleukin-2 (ProleukinH). A 2^{nd}-generation form of FGF-1 that is stable in the absence of heparin would eliminate a number of undesirable consequences associated with this additive. Further gains in utility (especially for wound healing application) could be realized if such mutants exhibit a PK profile providing extended in vivo elimination half-life or increased MRT.

FGF-1 has also been suggested for use in regenerating nervous system tissue following spinal cord injury or trauma, such as brachial plexus injury, neuroimmunologic disorders, such as acute or idiopathic transverse myelitis (TM), or any other disease or condition where regeneration and/or protection of neurons or neural tissue is desired, since FGF-1 is believed to stimulate neural proliferation and growth and may be neuroprotective [65, 66, 67].

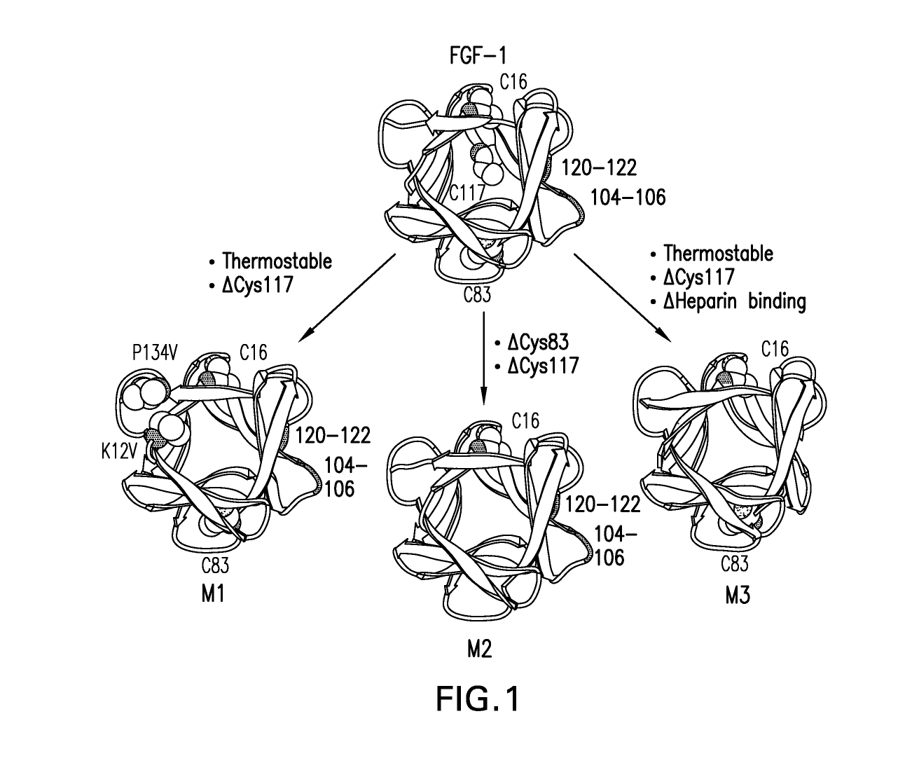

Described herein are genetically modified forms of human fibroblast growth factor-1 and methods for the treatment of ischemic diseases. A pharmokinetic (PK) study was performed on New Zealand White (NZW) rabbits, with human FGF-1 formulated in the presence and absence of heparin, as well as three mutant forms of FGF-1 (each formulated in the absence of heparin) (FIG. 1). These mutant forms were selected based upon their in vitro properties of increased thermostability [30] or reduced number of buried reactive thiols (these two properties cooperatively interact to significantly increase the in vitro functional half-life [31]). Another mutant, i.e., mutant M3 [SEQ ID NO: 4], is both thermostable and has deletions within the heparin-binding site that result in an order of magnitude reduction in heparin/heparan sulfate proteoglycan (HSPG) affinity [32]. A comparison of the PK parameters for these mutant forms permits an evaluation of the consequences upon the PK profile of heparin in the formulation of FGF-1, as well as the role of HSPG affinity in the overall distribution and elimination kinetics of FGF-1. The results support the hypothesis that the known HSPG sequestration of FGF-1 after intravenous (IV) bolus by liver, kidney and spleen is a key determinant of the PK distribution and elimination profile, and that heparin in the formulation of FGF-1 causes a more endocrine type profile. Enhancing the thermostability and eliminating buried reactive thiols yields a PK profile exhibiting greater efficiency of HSPG sequestration, resulting in less endocrine-type behavior, as well as extended elimination half-life and MRT. The aim of the present study was to determine if PK properties can be manipulated by specific protein mutation; the results show that they can, and that the mutations under study may have significant advantages over the WT FGF-1 protein for therapeutic application.

In FIG. 1, The FGF-1 structure (PDB accession 2AFG [62]) has three buried reactive thiols, namely C16, C83 and C117 and heparin-binding functionality associated with surface loops 104-106 and 120-122. Mutant M1 [SEQ ID NO: 2] (based upon PDB accession 2HWM for Lys12Val/Cys117Val mutant [30]) includes stabilizing mutations Lys12Val and Pro134Val (indicated) combined with elimination of one buried thiol (Cys117Val). Mutant M2 [SEQ ID NO: 3] (PDB accession 3FGM [31]) combines elimination of two buried thiols (Cys83Thr/Cys117Val) with two fully-buried stabilizing mutations (Leu44Phe/Phe132Trp; not shown) that offset the destabilizing effects of the Cys mutations (such that thermostability of M2 is equivalent to WT FGF-1). Mutant M3 [SEQ ID NO: 4] (PDB accession 303Q, a Phe108Tyr form of M3 that promotes crystallization [63]) has enhanced thermostability and elimination of one buried thiol (Cys117Val) and is thus similar to M1 [SEQ ID NO: 2]; however, M3 also has loop deletions (blue turn regions in FGF-1) that effectively eliminate heparin-binding functionality. In FIG. 1 the mutations indicated as “ΔCys117” and “ΔCys83” are not deletion mutations. Instead, these mutations involve substitution mutations, specifically Cys117Val and Cys83Thr mutations, respectively. This is in contrast to the label “Δ Heparin binding” which does involve deletion mutations (Δ120-122).

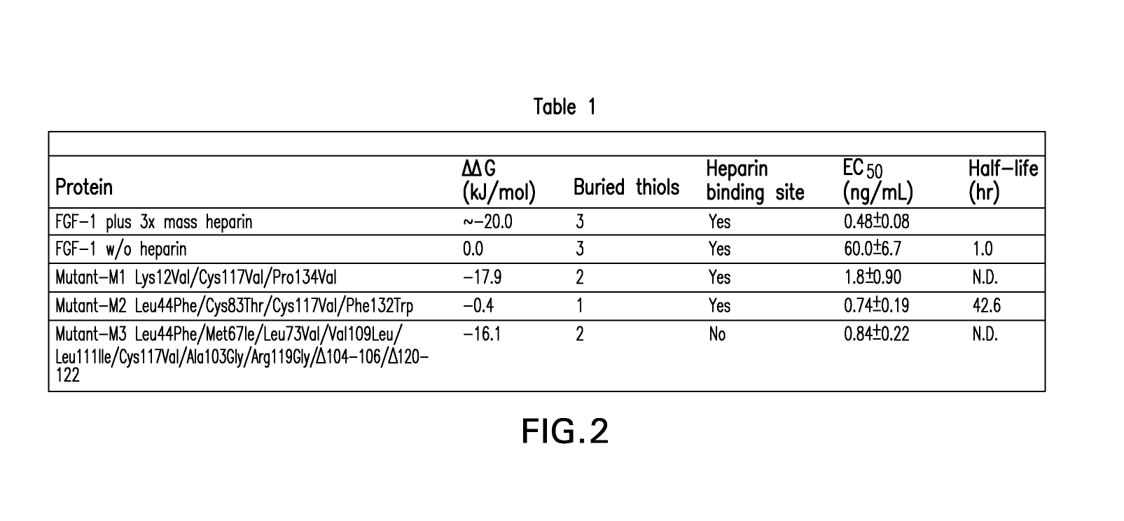

In Table 1 of FIG. 2, any negative ΔΔG value indicates increased thermostability compared to wild-type FGF-1. The EC₅₀ column lists values of effective concentrations for 50% maximum mitogenic activity against 3T3 fibroblasts and a lower value indicates increased effective mitogenic potency. The half-life is defined as residual mitogenic activity after cell culture medium incubation at 37° C. The profiles for: (1) FGF-1 plus 3-times mass heparin, (2) FGF-1 without heparin, (3) mutant M1 [SEQ ID NO: 2], (4) mutant M2 [SEQ ID NO: 3] and (5) mutant M3 [SEQ ID NO: 4] are derived from: (1) Copeland R A, Ji H, Halfpenny A J, Williams R W, Thompson K C, et al., “The structure of human acidic fibroblast growth factor and its interaction with heparin,” Archives of Biochemistry and Biophysics 289: 53-61 (1991) and Lee J, Blaber M, “The interaction between thermostability and buried free cysteines in regulating the functional half-life of fibroblast growth factor-1,” J. Mol. Bio.l 393: 113-127 (2009) (Lee et al.); (2) Lee et al. and Dubey V K, Lee J, Somasundaram T, Blaber S, Blaber M, “Spackling the crack: stabilizing human fibroblast growth factor-1 by targeting the N and C terminus beta-strand interactions,” J Mol Biol 371: 256-268 (2007) (Dubey et al.); (3) Dubey et al.; (4) Lee et al.; and (5) Brych S R, Dubey V K, Bienkiewicz E, Lee J, Logan T M, et al., “Symmetric primary and tertiary structure mutations within a symmetric superfold: a solution, not a constraint, to achieve a foldable polypeptide,” J. Mol. Biol. 344: 769-780 (2004), respectively.

Prior P K and imaging studies subsequent to IV bolus of radioiodinated or Technetium labeled FGF-1 in rodents have demonstrated a rapid clearance from plasma with corresponding accumulation in liver, kidney and spleen; and this accumulation correlates with the HSPG distribution in such tissues [53, 54, 55]. Increased tissue levels of HSPG restrict the distribution of signaling molecules such as FGF-1, can regulate concentration gradients of such signaling molecules, and may play a role in pattern formation in embryogenesis; conversely, decreased levels of HSPG promote long range transport of such signaling molecules [56]. Thus, HSPG binding is postulated to be a key determinant of the PK properties of FGF-1 [53, 54, 55]. The inclusion of mutants that differentially affect thermostability, number of buried reactive thiols, and heparin affinity, along with FGF-1±heparin, permits an analysis of the differential effects of such parameters upon FGF-1 PK properties. Inclusion of a form of FGF-1 (M3) with a heparin binding site deletion enables a direct experimental test of the role of HSPG in FGF-1 PK.

Comparison of FGF-1 with heparin and FGF-1 without heparin

The distribution half-life for FGF-1 with heparin is approximately twice as long as FGF-1 without heparin (10.2 min vs. 4.8 min, respectively). This result supports HSPG binding as a primary determinant of distribution kinetics for FGF-1 (as heparin occupying the heparin-binding site of FGF-1 must be competed off by HSPG in order for FGF-1 to bind to HSPG). In agreement with this interpretation, V₂/V₁ (a measure of the relative distribution of drug over the two compartments, with a larger ratio indicating a greater fraction of drug resides in the peripheral compartment) is three times greater (i.e., 6.2 versus 2.0) for FGF-1 without heparin compared to FGF-1 with heparin. The MRT of FGF-1 w/o heparin is also twice as great as compared to FGF-1 with heparin (i.e., 116 versus 59.9 min, respectively). The elimination half-life is essentially identical for FGF-16heparin and is consistent with identical PK profile subsequent to distribution (i.e., k₁₂ is principally determined by binding of FGF-1 to HSPG, which competes with bound heparin; whereas, k₂₁ is determined by release from HSPG). The increase in MRT for FGF-1 without heparin therefore appears due to the increased distribution in the peripheral compartment (i.e., more effective sequestration by HSPG).

Effect of Enhanced Thermostability; a Comparison of M1 and FGF-1 without Heparin

The heparin-binding site in FGF-1 is a consequence of the correctly folded structure of FGF-1. FGF-1 has low thermostability and is prone to denaturation [23, 24]; thus, stabilizing the structure might be expected to also confer a more stable heparin binding site with improved efficiency for HSPG binding. The M1 mutant [SEQ ID NO: 2] in comparison to FGF-1 without heparin exhibits an approximately two-fold shorter distribution half-life (i.e., 2.3 versus 4.8 min, respectively). Notably, while V₁ for M1 is essentially unaffected in comparison to FGF-1 w/o heparin, V₂ increases by a factor of four (and V₂/V₁ for M1 is 25.9 vs. 6.2 for FGF-1 w/o heparin); furthermore, the MRT for M1 doubles in value in comparison to FGF-1 without heparin. These effects with M1 appear due principally to a threefold increase in k₁₂ distribution constant, consistent with more efficient partitioning of M1 mutant from plasma to HSPG as well as reduction in kinetic constant of redistribution (i.e., k₂₁). In other words, the increase in thermostability (in addition to the loss of one buried reactive thiol) results in an apparent increased on-rate (k₁₂) and decreased off-rate (k₂₁) for HSPG. The elimination kinetic constant (k₁₀), postulated to be principally determined by kidney excretion, remains essentially unchanged.

Effect of Removal of Buried Reactive Thiols; a Comparison of M2 and FGF-1 without Heparin

FGF-1 contains three free cysteines that are chemically reactive and can participate in thiol chemistry (e.g., form mixed thiol adducts), resulting in irreversible unfolding and aggregate formation [31]. Reactivity of such thiols requires accessibility (i.e., protein unfolding) since these positions are buried within the protein core. Thus, there is a cooperative interplay between low thermostability and buried free thiols in the regulation of an irreversible unfolding pathway for FGF-1 [31]. Mutant M2 [SEQ ID NO: 3] eliminates two of the three buried reactive thiols in FGF-1 while maintaining equivalent thermostability to FGF-1. Elimination of these two reactive thiols results in a 40-fold longer in vitro functional half-life for mutant M2 in comparison to FGF-1 (Table 1 of FIG. 2). The overall PK rate constants of M2 and FGF-1 without heparin are similar, although there is a 1.8×-fold increase in elimination half-life. This appears to be due to a more efficient distribution (k₁₂), suggesting that chemical reactivity of free cysteine residues in FGF-1 may occur in plasma (with associated reduction of HSPG binding functionality). The M2 data is associated with larger errors than the other proteins; however, as with M1 there is a general increase in V₂ compared to FGF-1 without heparin and consistent with a more efficient overall binding of M2 to HSPG.

A direct test of the hypothesis that HSPG sequestration is the primary determinant of FGF-1 distribution kinetics could be performed using either an HSPG-deficient organism or a heparin-binding site-deficient FGF-1 (with the former likely being developmentally lethal). Mutant M3 has both enhanced thermostability and substantially diminished HSPG binding affinity (K_{d} for sucrose octasulfate is increased by an order of magnitude [32]). The stability increase of M3 is essentially identical to that of M1 (Table 1 of FIG. 2), and both have an identical single buried cysteine removed (Cys117); thus, a comparison of M3 and M1 can permit direct evaluation of HSPG affinity upon PK parameters. M3 exhibits a threefold longer distribution half-life and approximately fourfold shorter elimination half-life than M1. These results are consistent with HSPG affinity as a primary determinant of both distribution (k₁₂) and redistribution (k₂₁) kinetics of FGF-1. Notably, there is a greater than 10×-fold decrease in V₂N₁ for M3 in comparison with M1, principally due to a corresponding decrease in V₂. In this regard, the V₂N₁ value for M3 is essentially indistinguishable from that of FGF-1 with heparin; thus, confirming that bound heparin competes with HSPG for binding of FGF-1 to the peripheral compartment. These results also indicate that the peripheral compartment has a physical interpretation; namely, the HSPG in the vascular walls of kidney, liver and spleen [53, 54, 55].

### Initial Plasma Concentration

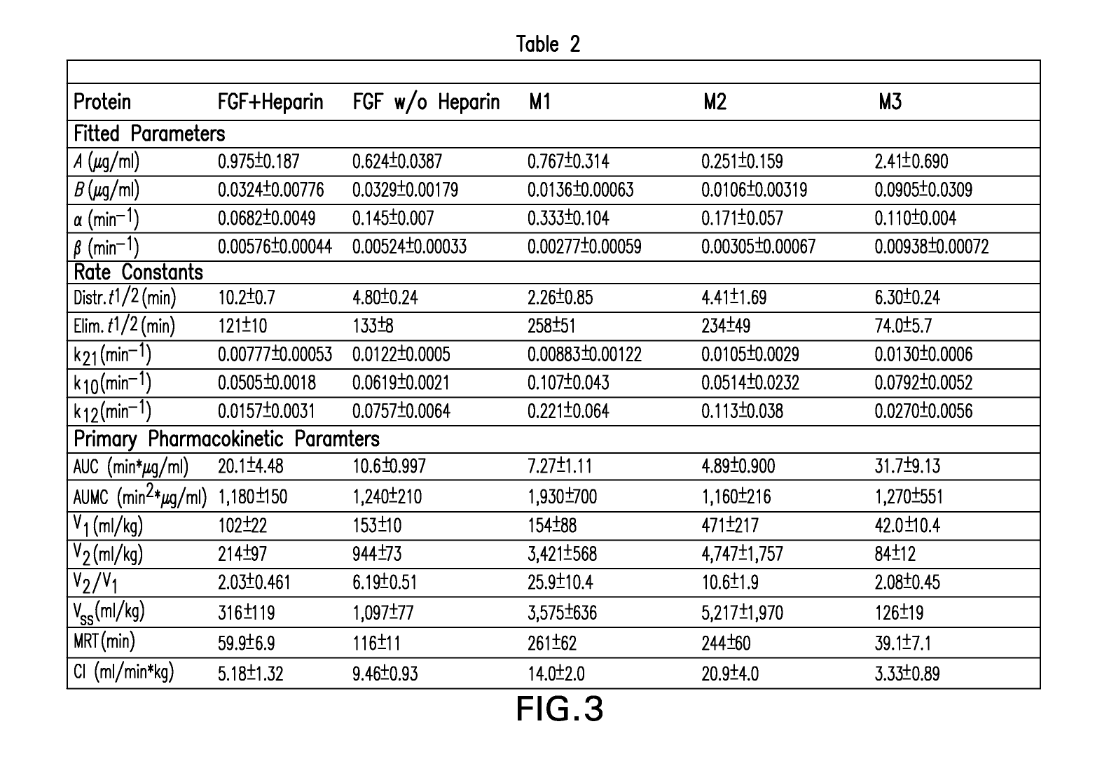

The maximum theoretical equilibrium blood concentration (i.e., assuming no distribution or elimination) in the present study is ˜2.00 μg/ml. A comparison of the extrapolated T₀ concentration (parameters A+B in Table 2 of FIG. 3) demonstrates essential agreement with this value only for the M3 mutant protein [SEQ ID NO: 4], with varying extent of diminished values for all other proteins. FGF-1 was analyzed with and without 3× mass excess of Heparin; mutant proteins were analyzed in the absence of heparin in each case. Specifically, the rank order of diminishing (A+B) values is M3, followed by FGF-1 with heparin (˜1.00 μg/ml), FGF-1 without heparin and mutant M1, (both ˜0.700 μg/ml), and mutant M2 (˜0.300 μg/ml). Since the ELISA standard curves utilize the respective mutant proteins, these values do not reflect errors in concentration measurement, rather, the effective removal from plasma of FGF-1 or mutant proteins within a single pass through the circulatory system. Since the initial state describes a non-equilibrium condition (e.g., redistribution rate=0) this discrepancy between (A+B) and theoretical maximum plasma concentration is interpreted as a substantial initial distribution to peripheral compartment during a single circulatory cycle. Even at comparatively high dosages of FGF-1, liver, kidney and spleen have been shown to quantitatively bind FGF-1 from plasma after only a single passage [53, 54, 55]. Thus, we interpret the rank order of discrepancy between (A+B) and maximum theoretical plasma concentration for FGF-1 and mutant proteins as principally based upon differential HSPG binding for these proteins. Thus, M3 with diminished heparin affinity has the greatest observed T₀ plasma concentration; followed by FGF-1 with heparin (where the bound heparin competes with HPSG for FGF-1 binding); then FGF-1 without heparin, M1 and M2.

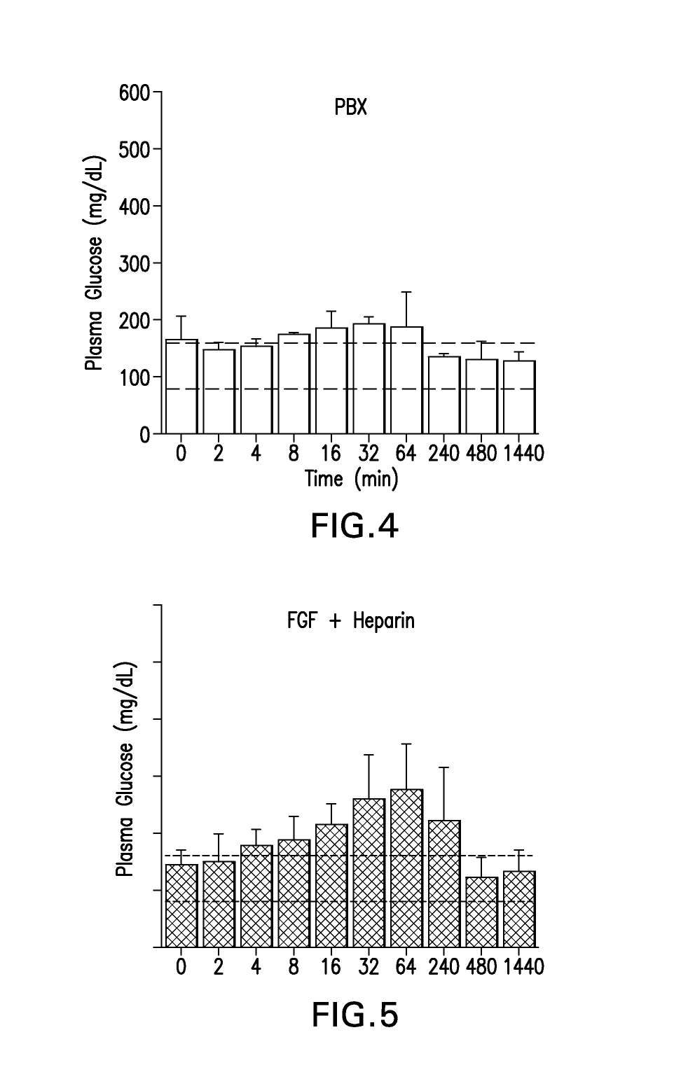

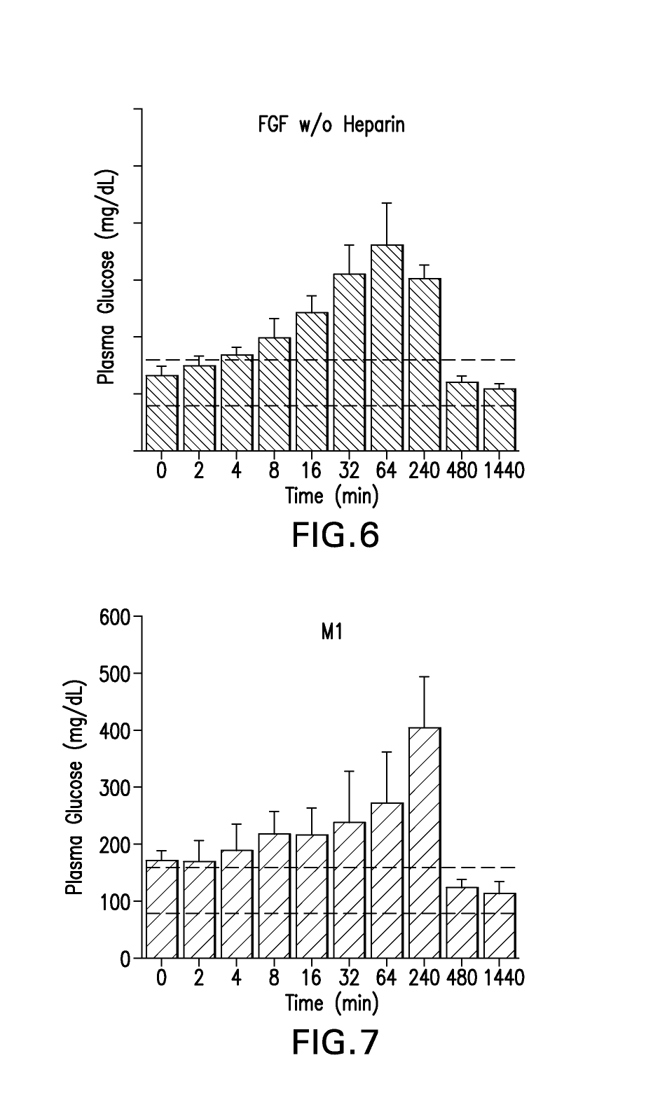

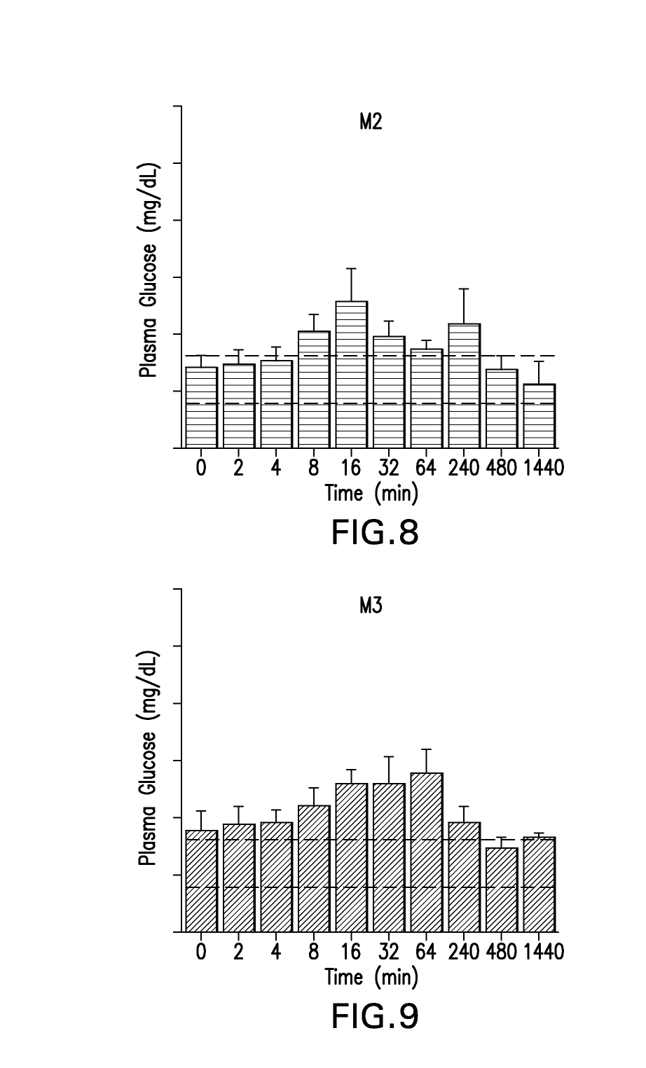

Overall, the present results provide strong experimental support for the hypothesis that HSPG binding serves as the principle physical basis of the peripheral compartment. This, in turn, identifies an important consequence upon PK properties when heparin is utilized in the formulation of FGF-1; specifically, heparin in the formulation will effectively increase the distribution half-life thereby promoting long range transport from the site of delivery and resulting in endocrine-like properties for the introduced FGF-1. The FGF family contains three members (FGF-19, 21 and 23) that lack a functional heparin-binding site and are referred to as “endocrine type” FGFs [57]. Due to their lack of HSPG binding, these FGFs circulate freely within the blood system, acting at a distance from the site of their synthesis. Thus, the addition of heparin to FGF-1 may promote mitogenic stimulation or angiogenesis distal to the site of delivery. Conversely, eliminating heparin favors local distribution, and may minimize unwanted endocrine type behavior. The endocrine type FGFs can act to reduce levels of plasma glucose and lipids [58, 59, 60, 61]. Although such effects have not been reported for FGF-1, plasma glucose and lipids were also evaluated as part of this PK study. Daily dosing at 1.0 mg/Kg of FGF-19 over a period of 7 days can reduce plasma glucose levels by 75 mg/dL [58]. In the present study a single IV bolus of 0.1 mg/Kg FGF-1±heparin resulted in an acute increase of plasma glucose levels of 150-200 mg/dL (FIGS. 4, 5, 6, 7, 8 and 9). The standard deviation for each protein measurement in FIGS. 4, 5, 6, 7, 8 and 9 is indicated by vertical error bar. The normal range for plasma glucose levels in NZW rabbits [50] is indicated by the two horizontal dashed lines. Thus, the effect of FGF-1 upon plasma glucose level is opposite to that observed with the endocrine FGFs, but is of a similar (or greater magnitude) and with a ten-fold lower dose (the chronic effects of repeated dosing of FGF-1 upon plasma glucose levels were not evaluated). To our knowledge, this activity of FGF-1 upon plasma glucose levels has not previously been reported. In the case of M2, the observed increase in plasma triglycerides also opposes the effect upon triglyceride levels observed for the endocrine FGFs. The endocrine FGF's may have uniquely different specificities for the different FGF receptors than FGF-1, and this may determine the observed differential effects upon plasma glucose and triglyceride levels.

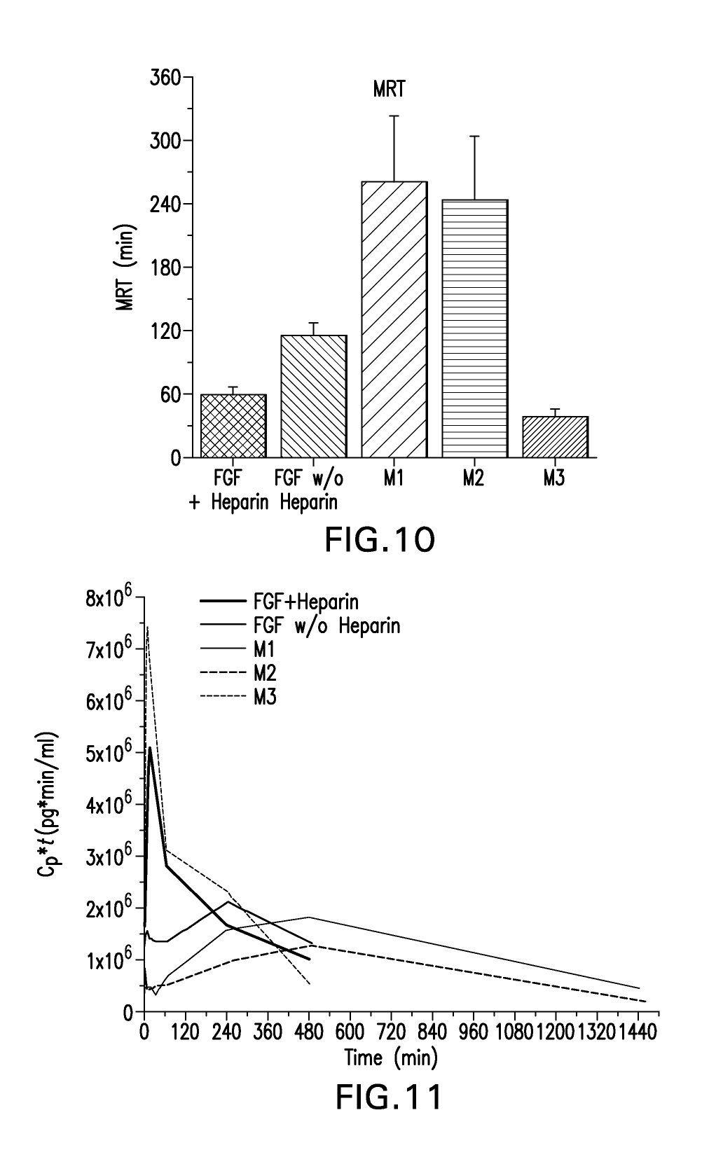

An increase in effective HSPG binding is observed for mutants M1 and M2, suggesting that mutational stabilization of the protein, or elimination of buried reactive thiols, can promote more effective HSPG binding. The PK data indicate that these mutational effects contribute to a shorter distribution half-life (i.e., reduced endocrine behavior), as well as an increased elimination half-life (due to slower redistribution kinetics from HSPG-bound protein), resulting overall in a more localized concentration at site of delivery as well as a longer MRT (FIGS. 3 and 10). As shown in FIG. 10, MRT is reduced for FGF-1+heparin, or for mutant M3 which has a diminished heparin binding site, showing that heparin sequestration is a prime determinant of MRT.

In effect, the efficient distribution of FGF-1 from plasma to HSPG (via the heparin binding site) stores FGF-1 for later redistribution into plasma (FIG. 11). In FIG. 11, efficient distribution of FGF-1 from plasma to HSPG via the heparin binding site serves as a storage reservoir for latent redistribution of FGF-1 from HSPG into plasma, extending MRT. Thus, the potential benefits of a mutant such as M1 or M2 over wild-type FGF-1 for therapeutic application (e.g., topical application for healing of diabetic ulcers) are multiple, and include the cost benefit in omitting heparin from formulation, safety benefit in elimination of heparin-associated side effects, safety benefit due to reduction in the potential for endocrine type mitogenic activity, therapeutic benefit in increased MRT, and cost and safety benefit in reduction in aggregation, improved storage potential, and potency upon reconstitution.

According to some embodiments of the present invention, the mutant FGF protein retains the ability to bind with specificity and affinity to a FGF receptor (FGFR) and trigger growth, proliferation, and/or survival of cultured and/or in vivo cells relative to untreated control cells (i.e., cells that are not exposed to a mutant FGF protein). Such cells may include, for example, fibroblast cells, neuronal or neuroblast cells, endothelial cells, chondrocytes, osteoblasts, myoblasts, smooth muscle cells, glial cells, etc., of human or animal origin known in the art to express one or more FGFRs or to respond to FGF proteins. See, e.g., Esch, F. et al., “Primary structure of bovine pituitary basic fibroblast growth factor (FGF) and comparison with the amino-terminal sequence of bovine brain acidic FGF,”PNAS USA 82(19):6507-11 (1985); and Gensburger, C. et al., “Effect of basic FGF on the proliferation of rat neuroblasts in culture,” C R Acad Sci III 303(11):465-468 (1986). Such a trigger of growth, proliferation, and/or survival of these cultured or in vivo cells may occur through binding and activation of a FGF receptor and downstream signaling within the cell(s). According to some embodiments, the mutant FGF protein of the present invention has a greater thermodynamic stability than wild-type human FGF-1. The mutant FGF protein of the present invention may have a greater thermodynamic stability than wild-type human FGF-1 if the mutant FGF protein has a ΔG_{unfolding} value greater than that of wild-type human FGF-1, such that ΔΔG=ΔG_{unfolding}(wild type)−ΔG_{unfolding}(mutant)<0, according to an isothermal equilibrium denaturation assay, differential scanning calorimetry assay, thermally-monitored spectroscopic assay, or other method of quantitation of ΔG_{unfolding} known in the art. According to some embodiments, the mutant FGF protein of the present invention has a greater functional half-life than wild-type human FGF-1 according to a cultured fibroblast proliferation assay. According to some embodiments, the mutant FGF protein of the present invention has essentially unaltered surface features relative to the wild type FGF protein and therefore has little or no immunogenic potential (i.e., the mutant FGF protein does not cause a significant immune reaction when introduced or administered to the body of an individual, subject, or patient).

According to some embodiments, a method of treatment of the present invention may employ a functional fragment of a mutant FGF protein, such as a functional fragment of a polypeptide sequence having at least 90% or 95% identity to at least a portion of the wild-type polypeptide sequence of one of the human FGF-1 protein forms. A functional fragment may be defined as a portion or fragment of a mutant FGF protein that retains FGF-like function. For example, a functional fragment may be a portion or fragment of a mutant FGF protein that is able to bind with specificity and affinity to at least one FGF receptor (FGFR) present on the surface of a cell. A functional fragment may also be a portion or fragment of a mutant FGF protein that is able to trigger growth, proliferation, and/or survival of cultured and/or in vivo cells relative to untreated control cells (i.e., cells that are not exposed to a mutant FGF protein). Such cells may include, for example, fibroblast cells, neuronal or neuroblast cells, endothelial cells, chondrocytes, osteoblasts, myoblasts, smooth muscle cells, glial cells, etc., of human or animal origin known in the art to express one or more FGFRs or respond to FGF proteins, which may occur through binding and activation of a FGF receptor and downstream signaling within the cell. The binding affinity and/or mitogenicity of a mutant FGF protein, or a functional fragment thereof, may be determined according to assays described herein or known in the art, such as a cultured fibroblast proliferation assay, etc.

According to some embodiments, a mutant FGF protein or a functional fragment used in a method of the present invention may further include any additional non-FGF peptide sequence or tag known in the art, which may be used to facilitate its detection or purification. For example, a mutant FGF protein may contain any additional non-FGF peptide sequence known in the art that provides an epitope or fluorescence for detection, such as Myc, HA, His, FLAG, GST, GFP, etc., or provides a basis for purification by chromatography. According to some embodiments, a mutant FGF protein or a functional fragment thereof that is described herein may further include an additional non-FGF peptide sequence or tag known in the art for targeting the mutant FGF protein to a particular tissue or cell, improved solubility, sustained activity or stability, improved expression, etc.

### EXAMPLES

### Example 1

### Mutant FGF-1 Selection

FGF-1 has poor thermostability and three buried reactive thiol groups (i.e., free cysteines) that cooperate to substantially limit the in vitro functional half-life (via an irreversible unfolding pathway) [23, 24, 31]. Point mutations that increase thermostability or effectively eliminate buried reactive thiols have been shown to significantly increase the in vitro functional half-life, enable full mitogenic potency in the absence of exogenously-added heparin, provide resistance to proteolysis, and reduce disulfide-mediated oligomerization/aggregation [30, 31]. Three mutations were selected to probe the effects of thermostability, buried reactive thiols, and heparin affinity upon the in vivo PK profile (Table 1 of FIG. 2). Mutant M1 is a thermostable mutant whose increase in thermostability approximates the stabilizing effect of bound heparin. M1 includes two point mutations (Lys12Val/Pro134Val) designed to stabilize the N- and C-terminus b-strand interactions, a region of structural weakness in the β-barrel architecture [30] (FIG. 1). M1 also includes a point mutation (Cys117Val) that eliminates one of three buried reactive thiols. Mutant M2 combines four point mutations; two mutations (Cys83Thr/Cys117Val) eliminate two of three buried reactive thiols in the structure (the other being Cys 16), and two mutations (Leu44Phe/Phe132Trp) are stabilizing core mutations that offset the destabilizing effects of the Cys mutations [31]. Thus, mutant M2 has thermostability indistinguishable from WT FGF-1, but has eliminated two buried reactive thiols. Mutant M3 combines eight point mutations and six deletion mutations and was developed to increase the overall threefold structural symmetry of FGF-1 [32, 33, 34]. The heparin binding site is a structural “aneurism” within the overall threefold tertiary structure symmetry characteristic of the b-trefoil fold [32, 35, 36] (FIG. 1). Deletion of the heparin binding site is associated with a marked increase in thermostability but loss of heparin binding functionality (thus supporting the “stability/function tradeoff” hypothesis [37, 38]). Therefore, M3 is similar to M1 in that both have an equivalent increase in thermostability (approximately equal to the effect of heparin addition to WT FGF-1) and both have the same buried reactive thiol eliminated (i.e., Cys117); however, whereas M1 has normal FGF-1 heparin binding site, M3 is deficient in this functionality. Thus, the set of FGF-1 with or without heparin and mutant proteins can effectively probe differential effects of thermostability, buried reactive thiols, and heparin/HSPG interaction upon the in vivo PK profile of FGF-1.

### Example 2

### Recombinant Protein Design

The 140 amino acid form of human FGF-1 [39] was utilized throughout this study. Although produced from an E. coli expression system, the FGF-1 protein was designed to be as close to the naturally occurring human form as possible. E. coli is known to initiate translation with an N-formyl methionine (f-Met) at the N-terminus of expressed proteins. Since f-Met can be recognized by the immune system of eukaryotes as foreign, a cleavable construct was designed to eliminate this prokaryotic modification. Specifically, the 140 amino acid form of FGF-1 was fused downstream of an enterokinase (EK) recognition sequence (Asp4 Lys) preceded by a flexible 20 amino acid linker (derived from the S-tag sequence of pBAC-3 and an N-terminal (His)₆ tag. The resulting expressed fusion protein utilizes the (His)₆ tag for efficient purification and is subsequently processed by EK digestion to yield the 140 amino acid form of FGF-1 having a native eukaryotic N-terminus

### Example 3

### Protein Preparation

Recombinant wild-type (WT) FGF-1 and mutant proteins were expressed from an E. coli host after induction with 10 mM isopropyl-β-D-thio-galactoside. The expressed protein was purified utilizing sequential column chromatography on Ni-nitrilo-triacetic acid (NTA) affinity resin followed by ToyoPearl HW-40S size exclusion chromatography. The purified protein was then digested with EK to remove the N-terminal (His)₆ tag, 20 amino acid linker, and (Asp4 Lys) EK recognition sequence. A subsequent second Ni-NTA chromatographic step was utilized to remove the released N-terminal peptide (along with any uncleaved fusion protein). Final purification (to ensure monodisperse FGF-1 protein) was per-formed using HiLoad Superdex 75 size exclusion chromatography equilibrated to 50 mM Na₂PO₄, 100 mM NaCl, 10 mM (NH₄)₂SO₄, 0.1 mM ethylenediaminetet-raacetic acid (EDTA), 5 mM L-Methionine, pH at 6.5 (“PBX” buffer); L-Methionine was included in PBX buffer to limit oxidization of reactive thiols and other potential oxidative degradation. An extinction coefficient of E₂₈₀ₙₘ (0.1%, 1 cm)=1.26 was utilized for concentration determination of FGF-1 [40, 41]; whereas, E₂₈₀ₙₘ=1.35 was used for M1, and E₂₈₀ₙₘ=1.31 was used for both M2 and M3 mutants (based upon the method of Gill and von Hippel [42]). For storage and use in all PK studies, the purified proteins were sterile filtered through a 0.22 micron filter, purged with N₂, snap frozen in dry ice and stored at −80° C. prior to use. The purity of the final proteins was assessed by both Coomassie Brilliant Blue and Silver Stain Plus stained sodium dodecylsulfate polyacrylamide gel electrophoresis (SDS PAGE). All proteins were prepared in the absence of heparin. Prior to IV bolus, heparin (3×mass), or PBX, was added to the WT FGF-1 protein (mutant proteins were diluted with PBX buffer only).

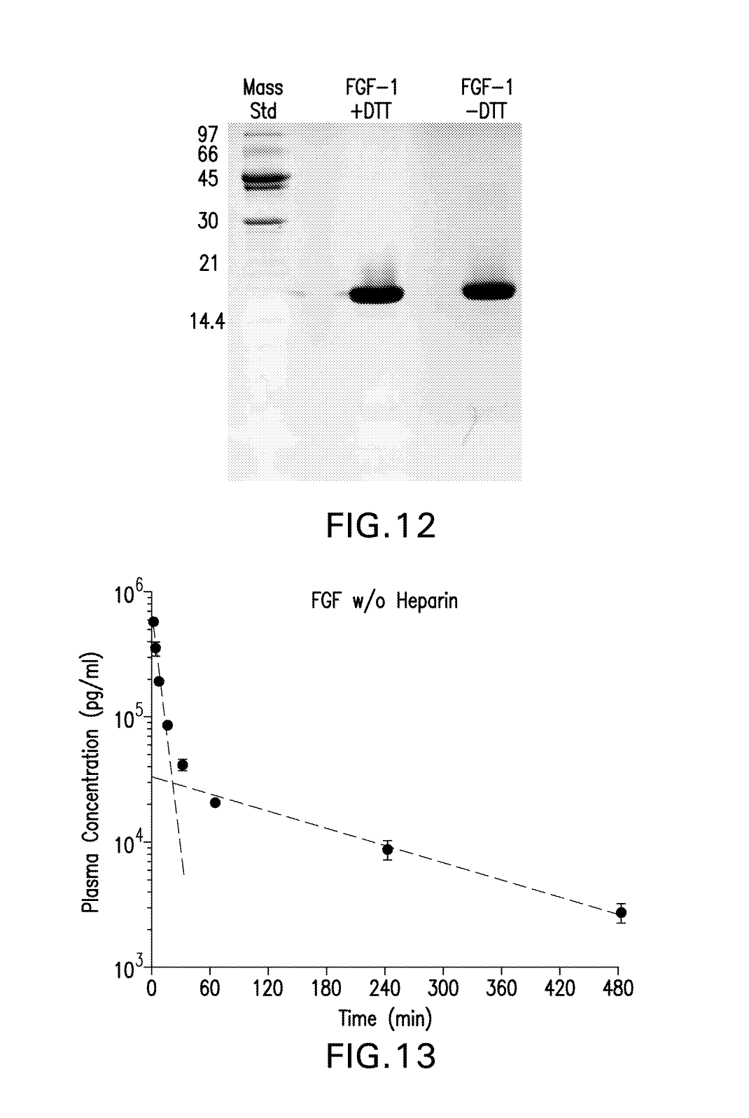

The wild-type FGF-1 and mutant proteins were isolated with >98% purity and homogenous monomeric forms, as judged from non-reduced silver stained SDS PAGE (FIG. 12). The overall yield of purified protein varied from 6-10 mg/L of E. coli cell culture, and a 1.0 L culture was sufficient to produce the necessary quantity of each protein for PK study.

### Example 4

### Pharmacokinetics

PK profiles were determined in 6-month old to 8-month old male NZW rabbits weighing 3.3-4.0 kg. Animals were housed in an AAALAC accredited facility in accordance with the Guide for the Care and Use of Laboratory Animals under a 12:12 hour light:dark cycle, 20° C. and 30-70% humidity. Rabbits were chosen for PK study due to fewer amino acid differences between human and rabbit FGF-1 (4 differences) than between human and mouse/rat (5 differences). Additionally, the rabbit hind limb model is a de facto standard for the study of induced ischemic disease and treatment by pro-angiogenic growth factors [43, 44, 45, 46]. Three rabbits per protein (n=3) were utilized to account for variation in individual response. Rabbits were sedated with 1.0 mg/kg equal dosage of butorphanol tartrate and acepromazine maleate through intramuscular injection and with 2% lidocaine jelly applied topically to the ears to facilitate injections and blood collection. A 3.0 ml pre-bleed (i.e., T=0 min) sample was taken from the right ear artery prior to IV bolus to establish baseline levels of FGF-1 and blood components. 100 mg/kg (330-400 mg per rabbit) of purified WT (+/23× mass heparin) or mutant FGF-1 protein (without heparin in each case) was administered (in 1.0 ml total volume, diluted with PBX) intravenously through the left ear marginal vein. Nominal 3.0 ml bleed volumes were collected using 23 gauge butterfly catheters from the right ear central artery into EDTA coated tubes. Whole blood was centrifuged at 4,000×g for 30 min and plasma was recovered, snap-frozen in dry ice, and stored at 280° C. prior to analysis. Plasma, rather than serum, was collected to avoid potential degradation of FGF-1 by activated coagulation proteases. Nominal bleeding time points were 1, 2, 4, 8, 16, 32, 64 min, 4, 8, and 24 hour post IV bolus; however, due to variations in animal response (shunting, coagulation, artery morphology, etc.) some variability in bleed time occurred. Exact times were recorded for each bleed collection and used in the analysis of pharmaco-kinetic profiles.

Three rabbits presented with either a hyperglycemic or hyperlipidemic condition in their pre-bleed sample and were excluded from the study. One rabbit presented with hypothermia during the study; additionally, several rabbits exhibited torpor subsequent to IV bolus that resulted in a reduced requirement for anesthesia prior to subsequent handling. However, no correlation could be identified between these general observations and a specific protein or PBX control. Plasma samples were visibly unremarkable with the exception of the 24 hour time point for mutant M2. For all rabbits given an IV bolus of mutant M2, the 24 hour time point exhibited a visible opacity (suggesting a possible hyperlipidemic condition). Opaque plasma samples were not observed for any earlier time point or with the 24 hour time point of any other mutant or wild-type FGF-1 protein. Therefore, additional 48 and 72 hour samples were collected for rabbits treated with mutant M2 for the purpose of visibly evaluating potential hyperlipidemia; subsequently, the cloudiness of the plasma resolved over these later time points. Because of this apparent hyperlipidemia, plasma triglyceride, cholesterol and liver panel assays were performed on all plasma samples.

The plasma concentration (Cp) of FGF-1 and mutant proteins was determined using a Quantikine™ human FGF-1 immuno-assay. This assay has been validated by the manufacturer for plasma samples collected in either EDTA or heparin-coated tubes (i.e., 0.1 mg/ml heparin in plasma); in the present study EDTA coated tubes were selected to avoid unwanted addition of heparin to the plasma samples. In the FGF-1+heparin IV bolus the maximum theoretical heparin concentration in plasma is ˜0.006 mg/ml; thus, this level of heparin, even in undiluted plasma samples, is within the manufacturer's validated conditions for the enzyme-linked immunosorbent assay (ELISA). The standard curve for each protein was established by a series of 1:2 dilutions (nominally 2,000-32 pg/ml) utilizing the purified wild-type or mutant FGF-1 proteins. This ELISA assay utilizes a polyclonal capture and detection antibody raised against full-length FGF-1 protein. However, all mutant proteins exhibited varying degree of reduced sensitivity in this ELISA. Postulating that stability effects or residual structure may be responsible, in part, for this reduced sensitivity, 4 M urea/phosphate buffered saline (PBS) was utilized as the assay diluent to promote full denaturation of proteins. This modification to the procedure improved sensitivity of the assay for these mutants, enabling a standard curve that spanned ˜4,000-100 pg/ml for M1, ˜2,000-50 pg/ml for M2, and ˜8,000-150 pg/ml for M3 (with all standard curves generated using 4M urea/PBS). The ELISA absorbance data were quantified using a Synergy H1 Hybrid Multi-Mode Microplate Reader. The early bleed time points had a much higher FGF-1 concentration than the maximum assayable ELISA concentration; thus, 1:10, 1:100, 1:1,000, and 1:10,000 dilutions for each plasma sample were initially performed to identify the appropriate dilution for quantitation within the assayable range of the ELISA. Subsequently, appropriately diluted plasma samples were assayed in quadruplicate. The standard curves of protein concentration vs. optical density for each ELISA were fitted using a logistic function (following manufacturer's instructions), and all plasma samples were quantified by interpolation using appropriate dilutions.

PK profiles for the Cp vs. time data were fit using the DataFit non-linear least squares fitting software package and a two-compartment pharmacokinetic model [47]:

Cp=Ae^{-αt}+Be^{-βt} (1)

where A and α define distribution phase kinetics and B and β define elimination phase kinetics, respectively. Macro rate constants were subsequently derived as follows:

Distribution t_{1/2}=0.693/α (2)

Elimination t_{1/2}=0.693/β (3)

Micro rate constants k₂₁ (redistribution rate constant), k₁₀ (elimination rate constant), and k₁₂ (distribution rate constant) were derived as follows:

k₂₁=(A×β+B×α)/(A+B) (4)

k₁₀=α×β/k₂₁ (5)

k₁₂=α+β−k₁₀−k₂₁ (6)

Primary pharmacokinetic parameters of Cl (clearance), V₁ (volume of the central compartment), and V₂ (volume of peripheral compartment) were derived as follows:

Cl=Dose/(A/α+B/β) (7)

V₁=Dose/(A+B) (8)

V₂=V₁×k₁₂/k₂₁ (9)

Other PK parameters, including AUC (area under the curve), V_{SS} (volume of distribution under steady state), AUMC (area under the first moment curve), and MRT were derived as follows:

AUC=(A/α+B/β) (10)

V_{SS}=(V₁×(k₁₂+k₂₁))/k₂₁ (11)

AUMC=V_{SS}+AUC²/Dose (12)

MRT=AUMC/AUC (13)

PK parameters for FGF-1 and mutant proteins were fit independently for each plasma concentration vs. time dataset, and the mean and standard deviation for each n=3 set were utilized in reporting the derived PK values.

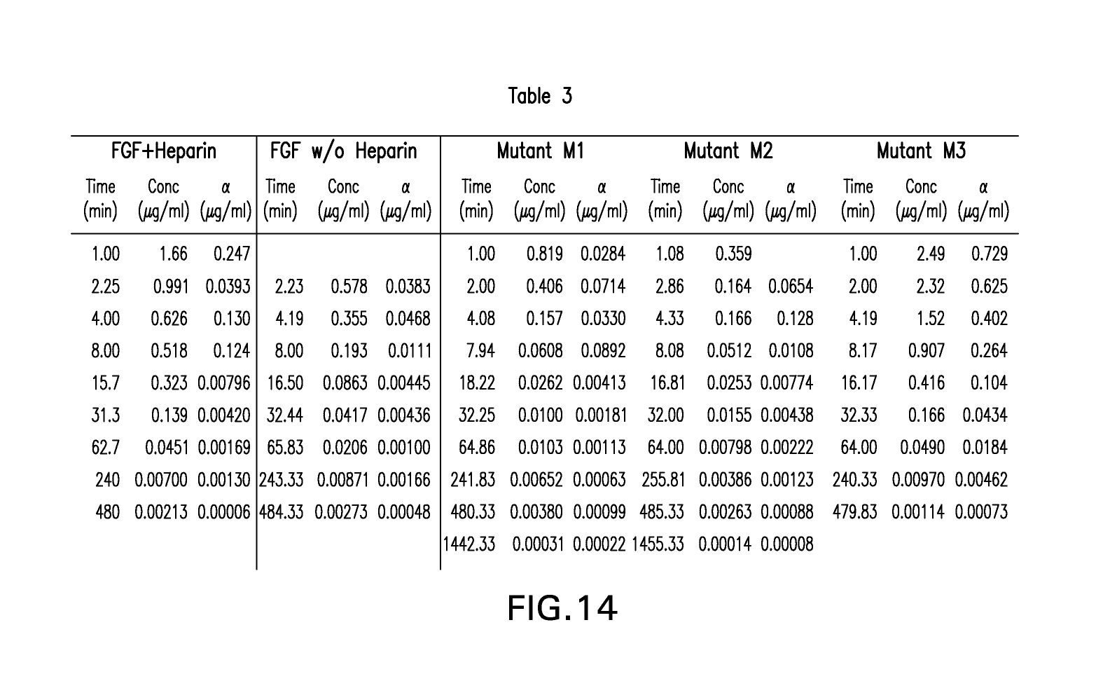

The FGF-1 ELISA assay can potentially cross-react with rabbit FGF-1; however, blood samples of the PBS control IV bolus, for every time point, and with each rabbit in this control set, yielded undetectable levels of endogenous FGF-1; thus, endogenous levels of rabbit FGF-1 were not an interfering factor. A plot of Log(Cp) vs. time for all data indicated a general bi-exponential decay that was in excellent agreement with a two-compartment model (FIG. 13). Non-linear least squares fitting of the Cp vs. time data demonstrated robust convergence of the fitted parameters regardless of variation of the initial values utilized in the fit. Additionally, deletion of the first or last data point in each set, followed by re-fitting, resulted in, 15% change in the refined parameters. Standard error for PK parameters of the n=3 set for each protein was approximately 15-20% with the exception of the M2 mutant, which yielded higher standard error values. The standard error of the fit to the two-compartment model for individual data sets was on the order of 10-15% (data not shown); thus, the standard error for the fit of the raw data to the model was approximately equivalent to the standard error between data sets. The derived PK constants are given in Table 2 FIG. 3 and a summary of the mean and standard deviation values for each protein is provided in Table 3 of FIG. 14.

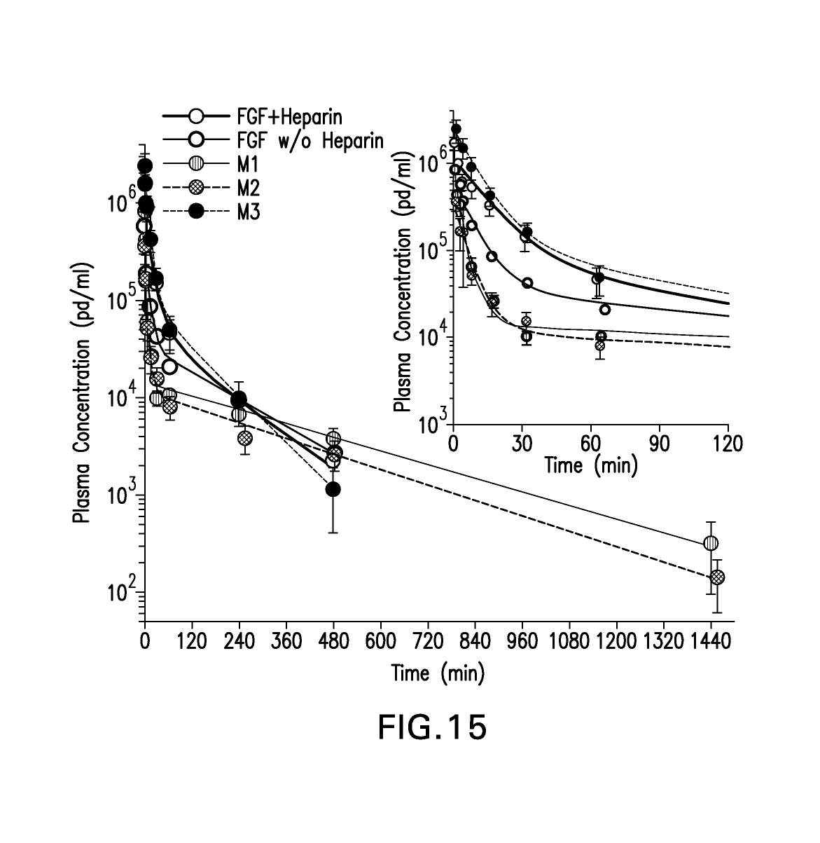

Based upon average NZW cardiac output (˜200 ml/min) and blood volume (˜6% mass, or ˜210 ml for a 3.5 kg rabbit) [48,49], a 1.0 min blood sample time point represents approximately a single pass of an IV bolus through the rabbit circulatory system and is therefore the earliest possible time for which homogenous plasma distribution can be considered. Analysis of the derived pharmacokinetic constants indicated that the 8 hour time point covered 3-5 elimination half-lives for all proteins except M1 and M2. For M1 and M2, data collection to 24 hour was required to cover 3-5 elimination half-lives; thus, PK analyses for all proteins met this requirement for two-compartment PK analysis [47] (Table 2 of FIG. 3). The 24 hour time point undiluted plasma samples for FGF-1 with or without heparin and M3 mutant proteins were below the detection limit of the ELISA assay; thus, the PK profile for these proteins was analyzed over the 480 min period. The mean PK time points with standard deviation (n=3) and fitted two-compartment functions for all proteins are provided in FIG. 15. As shown in FIG. 15, PBX IV bolus yielded no detectable endogenous FGF-1 for any time point (not shown). The inset on upper right shows a close-up of the 0-120 min time period. Standard deviation for each protein measurement is indicated by vertical error bar. The normal range for triglyceride levels in NZW rabbits [50] is indicated by the two horizontal dashed lines.

The general PK profile for the various FGF-1 proteins in this study follows a rapid distribution phase, involving ˜99% of the TO bolus concentration, and spanning approximately 20-60 min, prior to establishment of the pseudo-equilibrium elimination phase. The elimination phase for the various FGF-1 proteins could be followed (within the detection limits of the assay) over a period of 8-24 hour and involving another two-orders of magnitude reduction in plasma concentration. A notable variation of distribution and elimination kinetics was observed for the set of FGF-1 proteins, and in each case, there appeared to be a generally inverse correlation between the distribution and elimination kinetics (i.e., a fast distribution was followed by a slow elimination, and vice versa).

### Example 5

### Plasma Triglyceride, Cholesterol, Liver Chemistry, and Glucose Analyses

Plasma glucose values were measured using a model E4HD109 glucose-dehydrogenase pyrroloquinoline-quinone microfluidics glucose meter. Plasma glucose levels were measured in triplicate for each plasma sample time point.

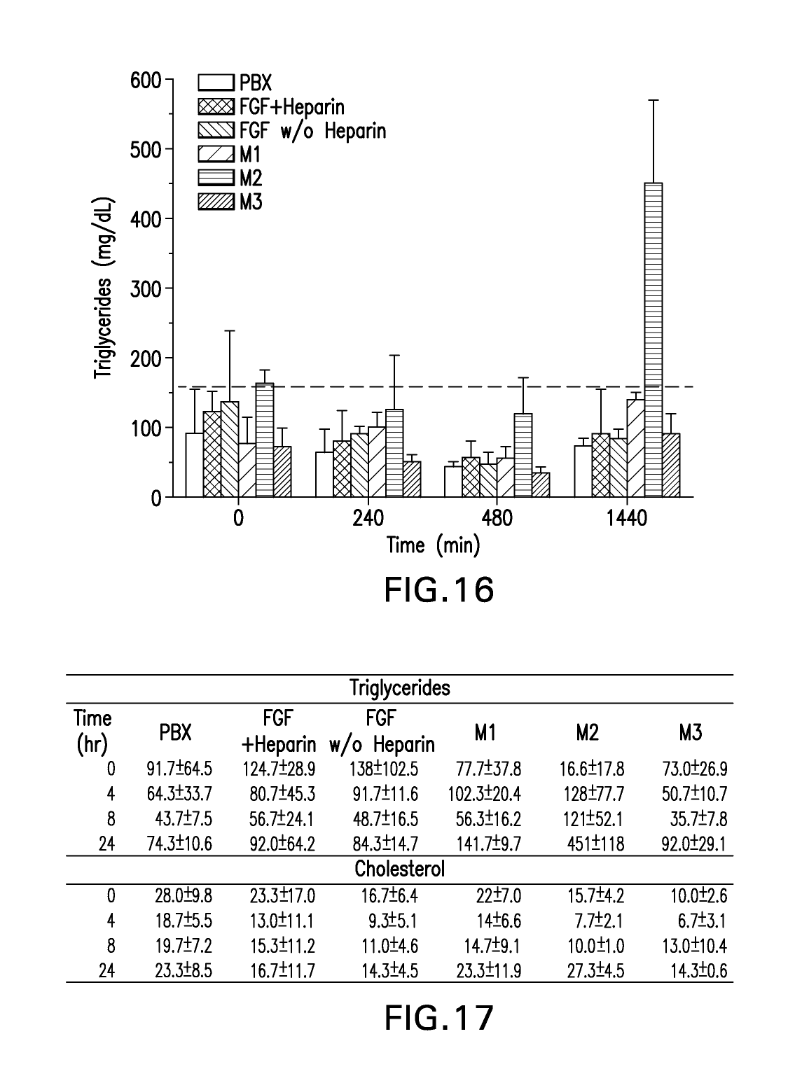

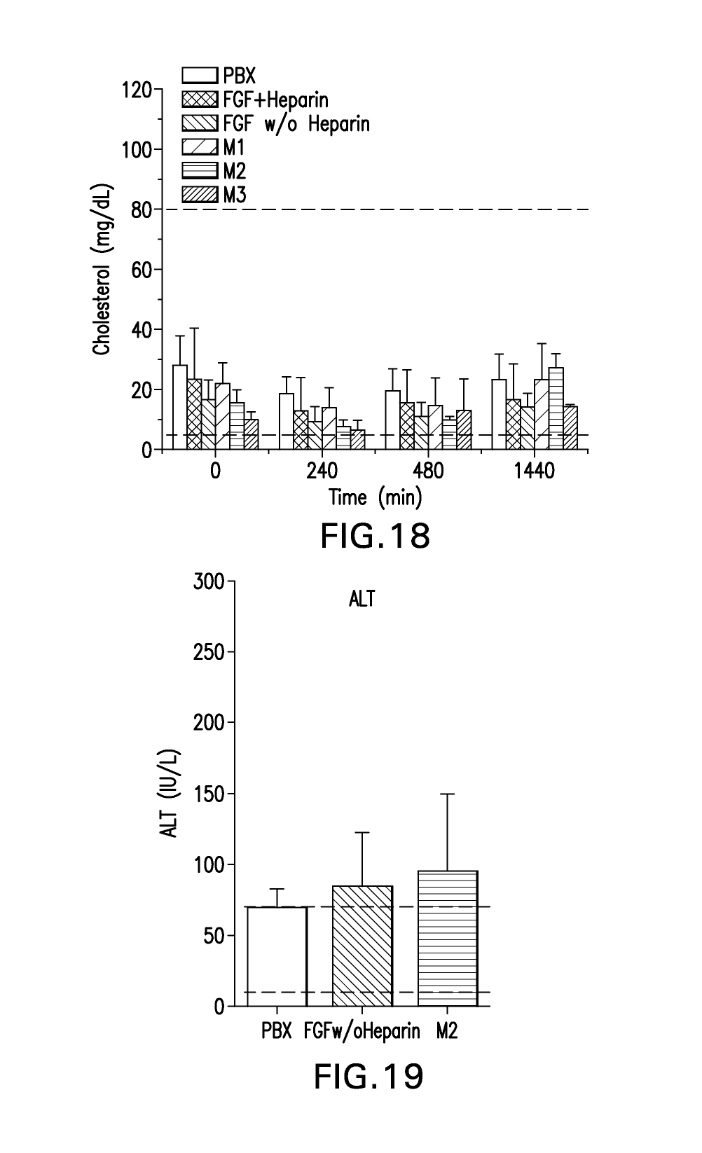

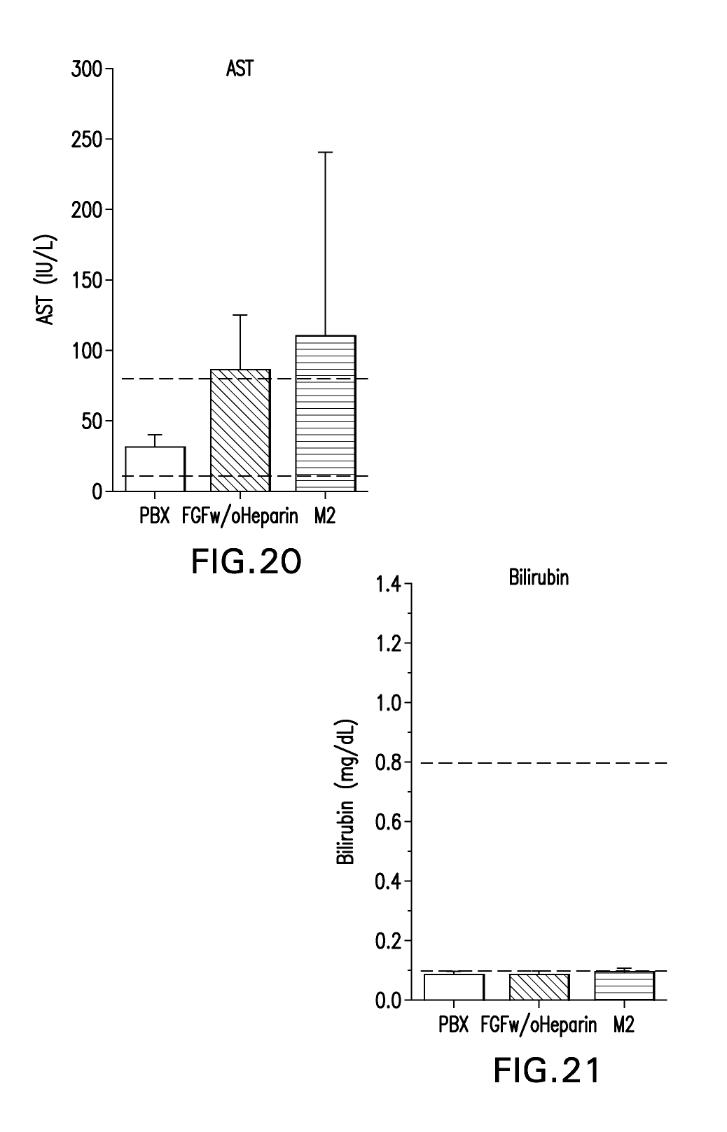

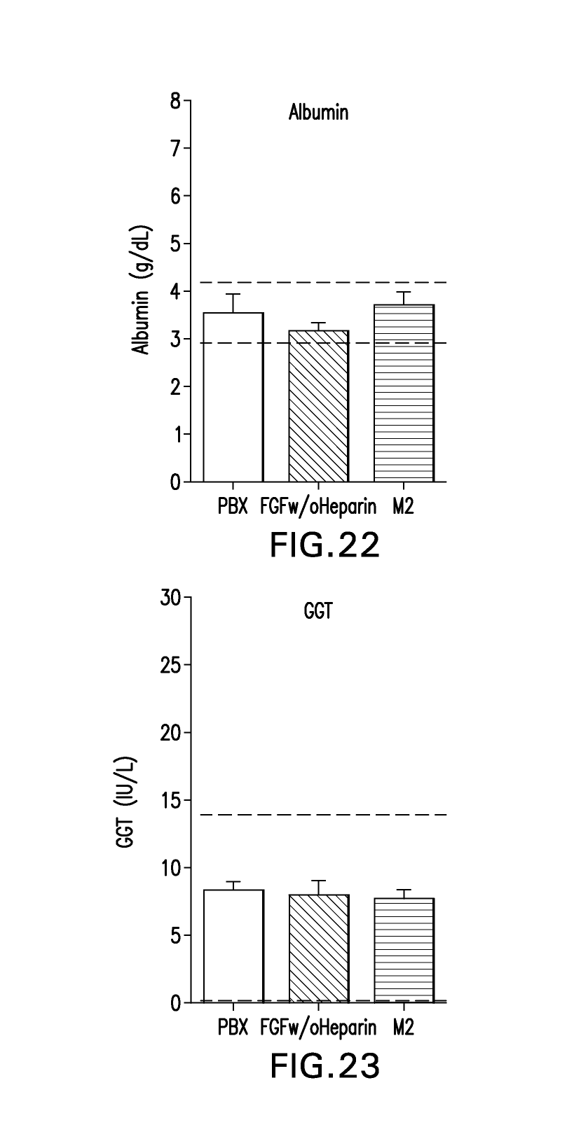

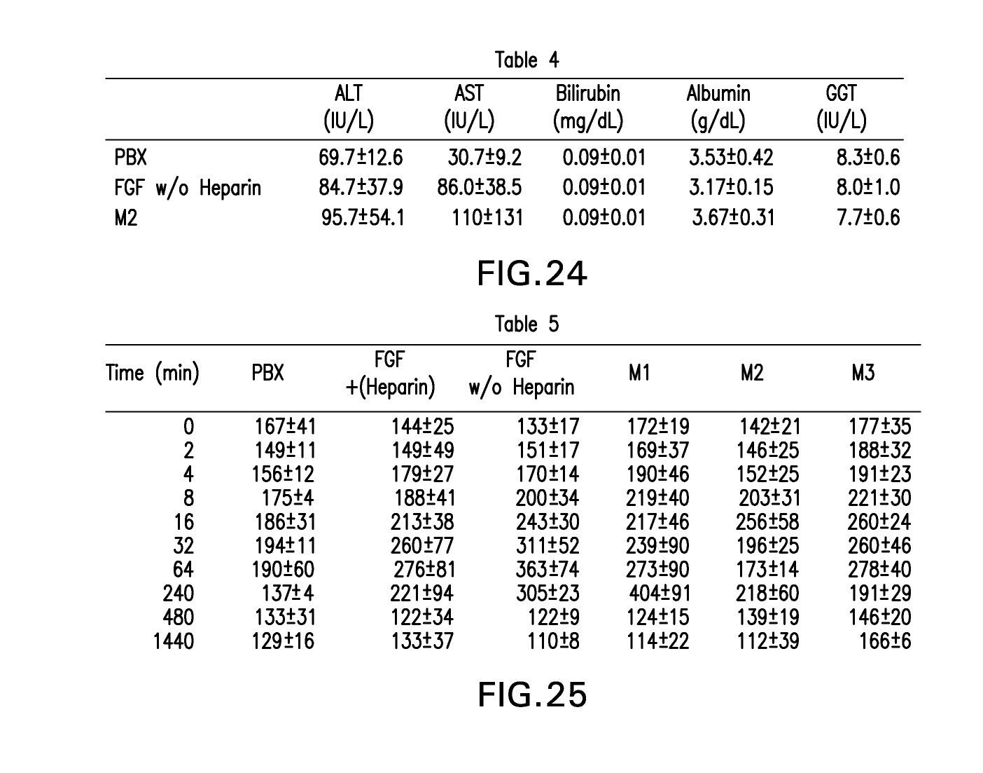

Plasma triglyceride levels resided within the normal range (15-160 mg/dL [50]) for NZW rabbits for all 0, 240, 480 and 1440 min (24 hour) time points with the exception of the 24 hour time point for mutant M2 (FIGS. 16 and 17). In FIG. 16, Standard deviation for each protein measurement is indicated by vertical error bar. The normal range for cholesterol levels in NZW rabbits [50] is indicated by the two horizontal dashed lines. This time point averaged 4516118 mg/dL, or approximately 3× the normal maximum range, indicating an acute hyperlipidemic condition 24 hour after IV bolus (which agreed with the visible observation of opaque plasma during blood collection). Plasma cholesterol levels fell within the normal range (5-8 mg/dL [50]) for NZW rabbits for all proteins and all time points (FIGS. 16 and 17). A liver chemistry profile was performed on the 24 hour plasma time point of the M2 mutant and compared to PBX control and FGF without heparin in an effort to identify any acute liver condition associated with the elevated triglycerides with the M2 24 hour plasma samples. Values for alanine transaminase (ALT), aspartate transaminase (AST), total bilirubin, albumin and c-glutamyltranspeptidase (GGT) were each within the normal range, although M2 samples exhibited comparatively large standard deviation (especially with ALT and AST values) (FIGS. 19, 20, 21, 22, 23 and 24). Table 4 of FIG. 24 shows 24 hour time point liver chemistry profiles that correspond with those shown in FIGS. 19, 20, 21, 22 and 23. EDTA from the blood collection tubes interferes with the alkaline phosphatase (ALP) assay, and so ALP values were not determined. In FIGS. 19, 20, 21, 22 and 23, the standard deviation for each protein measurement is indicated by vertical error bar. The normal range for cholesterol levels in NZW rabbits [50] is indicated by the two horizontal dashed lines.

Plasma glucose levels with the PBX control exhibited no statistically significant variation outside the normally expected range over the 24 hour evaluation period (FIGS. 4, 5, 6, 7, 8, 9 and 25). Table 5 of FIG. 25 shows values of plasma glucose levels (mg/dL) that correspond with those shown in FIGS. 4, 5, 6, 7, 8 and 9.

WT FGF-1 (both in the presence and absence of heparin) induced an apparent acute hyperglycemic condition, peaking at approximately 1 hour post-IV bolus. Mutant M1 exhibited a similar hyperglycemic condition, although the peak glucose level was observed at the later 4 hour time point. Mutant M2 exhibited a less significant hyperglycemia, while the effect upon plasma glucose for mutant M3 was essentially indistinguishable from that of FGF-1+heparin. In all cases, normal plasma glucose levels were restored by the 8 hour time point. Although the highest measured glucose value was 404691 mg/dL, the danger level for glucose in acute hyperglycemia in rabbits is considered 1,000 mg/dL [51,52].

The 100 mg/kg dose of FGF-1 or mutant proteins utilized in this PK study is considered to be a “high” dose [8, 9, 53, 54, 55]. The only significant event observed for the blood components assayed was elevated triglycerides in the 24 hour time point with the M2 mutant protein. This appears to be a transient hyperlipidemia that subsequently resolves over 48-72 hour. It is unclear what the cause of this hyperlipidemia is, since the mutations in the M2 protein are surface inaccessible and do not affect receptor or heparin binding properties, and the overall thermostability of M2 is essentially equivalent to WT FGF-1. Further study is required to understand the cause of the increased triglycerides and whether the effect is absent at lower doses.

All documents, patents, journal articles and other materials cited in the present application are incorporated herein by reference.

While the present invention has been disclosed with references to certain embodiments, numerous modification, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

### REFERENCES

The following references are referred to above and are incorporated herein by reference:
1. Levi-Montalcini R, “The Nerve Growth Factor,” Annals of the New York Academy of Sciences 118: 149-170 (1964).
2. Hoober J K, Cohen S, “Epidermal growth factor. 1. The stimulation of protein and ribonucleic acid synthesis in chick embryo epidermis,” Biochimica et Biophysica Acta 138: 347-356 (1967).
3. Schenkein I, Levy M, Bueker E D, Tokarsky E, “Nerve growth factor of very high yield and specific activity,” Science 159: 640-643 (1968).
4. Rudland P S, Seifert W, Gospodarowicz D, “Growth control in cultured mouse fibroblasts: induction of the pleiotypic and mitogenic responses by a purifed growth factor,” Proc Natl Acad Sci USA 71: 2600-2604 (1974).
5. Schumacher B, Stegmann T, Pecher P, “The stimulation of neoangiogenesis in the ischemic human heart by the growth factor FGF: first clinical results,” J Cardiovasc Surg (Torino) 39: 783-789 (1998).
6. Stegmann T J, Hoppert T, Schlurmann W, Gemeinhardt S, “First angiogenic treatment of coronary heart disease by FGF-1: long-term results after 3 years,” Cardiac and Vascular Regeneration 1: 5-9 (2000).
7. Wagoner L, Merrill W, Jacobs J, Conway G, Boehmer J, et al., “Angiogenesis protein therapy with human fibroblast growth factor (FGF-1): results of a phase I open label, dose escalation study in subjects with CAD not eligible for PCI or CABG,” Circulation 116, suppl. II: 443 (2007).
8. Ioli V, Wiens B, Mellin T, Thomas K, Ellis R, et al., “Effect of Topically Applied Acidic Fibroblast Growth Factor (FGF-1) on Healing of Chronic Venous Stasis and Diabetic Ulcers,” Abstracts of the 5th Annual Symposium on Advanced Wound Care: 150 (1992).
9. Mellin T N, Mennie R J, Cashen D E, Ronan J J, Capparella J, et al., “Acidic fibroblast growth factor accelerates dermal wound healing,” Growth Factors 7: 1-14 (1992).
10. Mellin T N, Cashen D E, Ronan J J, Murphy B S, DiSalvo J, et al., “Acidic fibroblast growth factor accelerates dermal wound healing in diabetic mice,” Journal of Investigative Dermatology 104: 850-855 (1995).
11. Bing M, Da-Sheng C, Zhao-Fan X, Dao-Feng B, Wei L, et al., “Randomized, multicenter, double-blind, and placebo-controlled trial using topical recombinant human acidic fibroblast growth factor for deep partial thickness burns and skin graft donor site,” Wound Repair and Regeneration 15:795-799 (2007).
12. Wang W, Lin S, Xiao Y, Huang Y, Tan Y, et al., “Acceleration of diabetic wound healing with chitosan-crosslinked collagen sponge containing recombinant human acidic fibroblast growth factor in healing-impaired STZ diabetic rats,” Life Sciences 82: 190-204 (2008).
13. Losordo D W, Vale P R, Symes J F, Dunnington C H, Esakof D D, et al., “Gene therapy for myocardial angiogenesis: initial clinical results with direct myocardial injection of phVEGF165 as sole therapy for myocardial ischemi,” Circulation 98: 2800-2804 (1998).
14. Rosengart T K, Lee L Y, Patel S R, Sanborn T A, Parikh M, et al., “Angiogenesis gene therapy: phase I assessment of direct intramyocardial administration of an adenovirus vector expressing VEGF121 cDNA to individuals with clinically significant severe coronary artery disease,” Circulation 100: 468-474 (1999).
15. Hedman M, Hartikainen J, Syvanne M, Stjernvall J, Hedman A, et al., “Safety and feasibility of catheter-based local intracoronary vascular endothelialgrowth factor gene transfer in the prevention of postangioplasty and in-stentrestenosis and in the treatment of chronic myocardial ischemia: phase II results of the Kuopio Angiogenesis Trial (KAT),” Circulation 107: 2677-2683 (2003).
16. Schumacher B, Pecher P, von Specht B U, Stegmann T, “Induction of neoangiogenesis in ischemic myocardium by human growth factors: first clincal results of a new treatment of coronary heart disease,” Circulation 97: 645-650 (1998).
17. Landau C, Jacobs A K, Haudenschild C C, “Intrapericardial basic fibroblast growth factor induces myocardial angiogenesis in a rabbit model of chronic ischemia,” American Heart Journal 129: 924-931 (1995).
18. Laham R J, Rezaee M, Post M, Novicki D, Sellke F W, et al., “Intrapericardial delivery of fibroblast growth factor-2 induces neovascularization in a porcine model of chronic myocardial ischemia,” J Pharmacol Exp Ther 292: 795-802 (2000).
19. Pierce G F, Mustoe T A, Altrock B W, Deuel T F, Thomason A, “Role of platelet-derived growth factor in wound healing,” J Cell Biochem 45: 319-326 (1991).
20. Brown R L, Breeden M P, Greenhalgh D G, “PDGF and TGF-an act synergistically to improve wound healing in the genetically diabetic mouse,” J Surg Res 56: 562-570 (1994).
21. Jimenez P A, Rampy M A, “Keratinocyte growth factor-2 accelerates wound healing in incisional wounds,” J Surg Res 81: 238-242 (1999).
22. Marti G P, Mohebi P, Liu L, Wang J, Miyashita T, et al., “KGF-1 for wound healing in animal models,” Methods Mol Biol 423: 383-391 (2008).
23. Blaber S I, Culajay J F, Khurana A, Blaber M, “Reversible thermal denaturation of human FGF-1 induced by low concentrations of guanidine hydrochloride,” Biophysical Journal 77: 470-477 (1999).
24. Copeland R A, Ji H, Halfpenny A J, Williams R W, Thompson K C, et al., “The structure of human acidic fibroblast growth factor and its interaction with heparin,” Archives of Biochemistry and Biophysics 289: 53-61 (1991).
25. Alsenaidy M A, Wang T, Kim J H, Joshi S B, Lee J, et al., “An empirical phase diagram approach to investigate conformational stability of “secondgeneration” functional mutants of acidic fibroblast growth factor (FGF-1),” Protein Sci 21: 418-432 (2012).
26. Seitz C S, Brocker E B, Trautman A, “Management of allergy to heparins in postoperative care: subcutaneous allergy and intravenous tolerance,” Dermatology 14: 4 (2008).
27. Prechel M, Walenga J M, “Heparin-induced thrombocytopenia: an update,” Seminars in Thrombostasis and Hemostasis 38: 483-496 (2012).
28. Matuszewska B, Keogan M, Fisher D M, Soper K A, Hoe C-M, et al., “Acidic fibroblast growth factor: evaluation of topical formulations in a diabetic mouse wound healing model,” Pharmaceutical Research 11: 65-71 (1994).
29. Kurtzman A L, Govindarajan S, Vahle K, Jones J T, Heinrichs v, et al., “Advances in directed protein evolution by recursive genetic recombination: applications to therapeutic proteins,” Curr Opin Biotech 12: 361-370 (2001).
30. Dubey V K, Lee J, Somasundaram T, Blaber S, Blaber M, “Spackling the crack: stabilizing human fibroblast growth factor-1 by targeting the N and C terminus beta-strand interactions,” J Mol Biol 371: 256-268 (2007).
31. Lee J, Blaber M, “The interaction between thermostability and buried free cysteines in regulating the functional half-life of fibroblast growth factor-1,” Journal of Molecular Biology 393: 113-127 (2009).
32. Brych S R, Dubey V K, Bienkiewicz E, Lee J, Logan T M, et al., “Symmetric primary and tertiary structure mutations within a symmetric superfold: a solution, not a constraint, to achieve a foldable polypeptide,” J Mol Biol 344: 769-780 (2004).
33. Brych S R, Blaber S I, Logan T M, Blaber M, “Structure and stability effects of mutations designed to increase the primary sequence symmetry within the core region of a b-trefoil,” Protein Science 10: 2587-2599 (2001).
34. Brych S R, Kim J, Logan T M, Blaber M, “Accommodation of a highly symmetric core within a symmetric protein superfold,” Protein Science 12: 2704-2718 (2003).
35. McLachlan A D, “Three-fold structural pattern in the soybean trypsin inhibitor (Kunitz),” Journal of Molecular Biology 133: 557-563 (1979).
36. Murzin A G, Lesk A M, Chothia C, β-Trefoil fold. Patterns of structure and sequence in the kunitz inhibitors interleukins-1β and 1α and fibroblast growth factors,” Journal of Molecular Biology 223: 531-543 (1992).
37. Beadle B M, Shoichet B K, “Structural basis of stability—function tradeoffs in enzymes,” Journal of Molecular Biology 321: 285-296 (2002).
38. Tokuriki N, Stricher F, Serrano L, Tawfik D S, “How protein stability and new functions trade off,” PLoS Comput Biol 4: e1000002 (2008).
39. Gimenez-Gallego G, Conn G, Hatcher V B, Thomas K A, “The complete amino acid sequence of human brain-derived acidic fibroblast growth factor,” Biochemical and Biophysical Research Communications 128: 611-617 (1986).
40. Tsai P K, Volkin D B, Dabora J M, Thompson K C, Bruner M W, et al., “Formulation design of acidic fibroblast growth factor,” Pharmaceutical Research 10: 649-659 (1993).
41. Zazo M, Lozano R M, Ortega S, Varela J, Diaz-Orejas R, et al., “High-level synthesis in Escherichia coli of a shortened and full-length human acidic fibroblast growth factor and purification in a form stable in aqueous solutions,” Gene 113: 231-238 (1992).
42. Gill S C, von Hippel P H, “Calculation of protein extinction coefficients from amino acid sequence data,” Anal Biochem 182: 319-326 (1989).
43. Little R A, “Local and general responses to injury in the newborn rabbit,” Postgrad Med J 45: 559-561 (1969).
44. Baffour R, Berman J, Garb J L, Rhee S W, Kaufman J, et al., “Enhanced angiogenesis and growth of collaterals by in vivo administration of recombinant basic fibroblast growth factor in a rabbit model of acute lower limb ischemia: dose-response effect of basic fibroblast growth factor,” Journal of Vascular Surgery 16: 181-192 (1992).
45. Pu L-Q, Sniderman A D, Brassard R, Lachapelle K J, Graham A M, et al., “Enhanced revascularization of the ischemic limb by angiogenic therapy,” Circulation 88: 208-215 (1993).
46. Pu L-Q, Jackson S, Lachapelle K J, Arekat Z, Graham A M, et al., “A persistent hindlimb ischemia model in rabbit,” Journal of Investigative Surgery 7:49-60 (1994).
47. Rosenbaum S, “Basic pharmacokinetics and pharmacodynamics—an integrated textbook and computer simulations,” Hoboken: John Wiley & Sons, Inc (2011).
48. Hgashiyama A, Watkins M W, Chen Z, LeWinter M M, “Effects of EMD 57033 on Contraction and Relaxation in Isolated Rabbit Hearts,” Circulation 92: 3094-3104 (1995).
49. Preckel B, Schlack W, Heibel T, Rutten H, “Xenon produces minimal haemodynamic effects in rabbits with chronically compromised left ventricular function,” British Journal of Anaesthesia 88: 264-269 (2002).
50. Evans GO, editor, “Animal clinical chemistry—a practical handbook for toxicologists and biomedical researchers,” Boca Raton: Taylor & Francis Group LLC (2009).
51. Thrall M A, Baker D C, Campbell T W, DeNicola D B, Fettman M J, et al., “Veterinary hematology and clinical chemistry,” Hoboken: Wiley Blackwell Publishing (2004).
52. Willard M D, Tvedten H, “Small animal clinical diagnosis by laboratory methods,” Philadelphia: W.B. Saunders Co (2007).
53. Rosengart T K, Kuperschmid J P, Maciag T, Clark R E, “Pharmacokinetics and distribution of heparin-binding growth factor-1 (endothelial cell growth factor) in the rat,” Circulation Research 64: 227-234 (1989).
54. Hondermarck H, Courty J, Boilly B, Thomas D, “Distribution of intravenously administered acidic and basic fibroblast growth factors in mouse,” Experientia 46: 973-974 (1990).
55. Zinn K R, Kelpke S, Chaudhuri T R, Sugg T, Mountz J M, et al., “Imaging Tc-99m-labeled FGF-1 targeting in rats,” Nuclear Medicine & Biology 27: 407-414 (2000).
56. Hacker U, Nybakken K, Perrimon N, “Heparan sulphate proteoglycans: the sweet side of development,” Nature Rev 6: 530-541 (2005).
57. Beenken A, Mohammadi M, “The FGF family: biology, pathophysiology and therapy,” Nature Rev 8: 235-253 (2009).
58. Fu L, John L M, Adams S H, Yu X X, Tomlinson E, et al., “Fibroblast growth factor 19 increases metabolic rate and reverses dietary and leptin-deficient diabetes,” Endocrinology 145: 2594-2603 (2004).
59. Kharitonenkov A, Shiyanova T L, Koester A, Ford A M, Micanovic R, et al., “FGF-21 as a novel metabolic regulator,” J Clin Invest 115: 1627-1635 (2005).
60. Ryden M, “Fibroblast growth factor 21: an overview from a clinical perspective,” Cell Mol Life Sci 66: 2067-2073 (2009).
61. Reiche M, Bachmann A, Lossner U, Bluher M, Stumvoll M, et al., “Fibroblast growth factor 19 serum levels: relation to renal function and metabolic parameters,” Horm Metab Res 42: 178-181 (2010).
62. Blaber M, DiSalvo J, Thomas K A, “X-ray crystal structure of human acidic fibroblast growth factor,” Biochemistry 35: 2086-2094 (1996).
63. Lee J, Blaber S I, Dubey V K, Blaber M, “A polypeptide “building block” for the β-trefoil fold identified by “top-down symmetric deconstruction”,” J Mol Biol 407: 744-763 (2011).
64. Hewitt C D, Innes D J, Savory J, Wills M R, “Normal biochemical and hematological values in New Zealand White rabbits,” Clinical Chemistry 35:1777-1779 (1989).
65. Lin, P H, Cheng, H, Huang, W C, Chuang, T Y, “Spinal cord implantation with acidic fibroblast growth factor as a treatment for root avulsion in obstetric brachial plexus palsy,” J Chin Med Assoc 68:392-396 (2005).
66. Cheng, H, Liao, K K, Liao, S F, Chuang, T Y, Shih, Y H, “Spinal cord repair with acidic fibroblas growth factor as a treatment for a patient with chronic paraplegia,” SPINE 29″ E284-E288 (2004).
67. Lin, P H, Chuang, T Y, Liao, K K, Cheng, H, Shih, Y S, “Functional recovery of chronic complete idiopathic transverse myelitis after administration of neurotrophic factors,” Spinal Cord 44:254-257 (2006).

What is claimed is:

1. A method, comprising the following step:
(a) administering to an individual having an ischemic condition or disease a composition comprising a mutant fibroblast growth factor (FGF) protein having a polypeptide sequence that is at least 90% identical to the polypeptide sequence of wild-type human FGF-1 protein (SEQ ID NO: 2),
wherein the numbering of the amino acid positions is based on the numbering scheme for the 140 amino acid form of human FGF-1, and
wherein the composition is administered without heparin.

2. The method of claim 1, wherein the mutant FGF protein has a polypeptide sequence that is at least 95% identical to the polypeptide sequence of wild-type human FGF-1 protein (SEQ ID NO: 2).

3. The method of claim 1, wherein the mutant FGF protein binds specifically to at least one FGF receptor (FGFR) and triggers growth or proliferation of cultured fibroblast cells.

4. The method of claim 1, wherein the mutant FGF protein has a greater functional half-life than wild-type human FGF-1 according to a cultured fibroblast proliferation assay.

5. The method of claim 1, wherein the protein structure of the mutant FGF protein has a greater thermodynamic stability than wild-type human FGF-1.

6. The method of claim 5, wherein the mutant FGF protein has a ΔG_{unfolding} value greater than that of wild-type human FGF-1 such that
ΔΔG=ΔG_{unfolding}wild-type−ΔG_{unfolding}mutant<0
according to one or more of the following methods: isothermal equilibrium denaturation, differential scanning calorimetry, or temperature-dependent spectroscopy.

7. The method of claim 1, wherein the ischemic condition or disease is coronary artery disease or peripheral vascular disease.

8. The method of claim 1, wherein the composition is administered locally at or near a site of the ischemic condition or disease within a body of the individual.

9. The method of claim 1, wherein the composition is administered locally at or near a site within a body of the individual causing the ischemic condition or disease.

10. The method of claim 9, wherein the composition is administered locally at or near an occluded blood vessel.

11. A method, comprising the following step:
(a) administering to an individual having an ischemic condition or disease a composition comprising a mutant fibroblast growth factor (FGF) protein having a polypeptide sequence that is at least 90% identical to the polypeptide sequence of wild-type human FGF-1 protein (SEQ ID NO: 3),
wherein the numbering of the amino acid positions is based on the numbering scheme for the 140 amino acid form of human FGF-1, and
wherein the composition is administered without heparin.

12. The method of claim 11, wherein the mutant FGF protein has a polypeptide sequence that is at least 95% identical to the polypeptide sequence of wild-type human FGF-1 protein (SEQ ID NO: 3).

13. The method of claim 11, wherein the mutant FGF protein binds specifically to at least one FGF receptor (FGFR) and triggers growth or proliferation of cultured fibroblast cells.

14. The method of claim 11, wherein the mutant FGF protein has a greater functional half-life than wild-type human FGF-1 according to a cultured fibroblast proliferation assay.

15. The method of claim 11, wherein the protein structure of the mutant FGF protein has a greater thermodynamic stability than wild-type human FGF-1.

16. The method of claim 15, wherein the mutant FGF protein has a ΔG_{unfolding} value greater than that of wild-type human FGF-1 such that
ΔΔG=AG_{unfolding}wild-type−ΔG_{unfolding}mutant<0
according to one or more of the following methods: isothermal equilibrium denaturation, differential scanning calorimetry, or temperature-dependent spectroscopy.

17. The method of claim 11, wherein the ischemic condition or disease is coronary artery disease or peripheral vascular disease.

18. The method of claim 11, wherein the composition is administered locally at or near a site of the ischemic condition or disease within a body of the individual.

19. The method of claim 11, wherein the composition is administered locally at or near a site within a body of the individual causing the ischemic condition or disease.

20. The method of claim 19, wherein the composition is administered locally at or near an occluded blood vessel.

21. A method, comprising the following step:
(a) administering to an individual having an ischemic condition or disease a composition comprising a mutant fibroblast growth factor (FGF) protein having a polypeptide sequence that is at least 90% identical to the polypeptide sequence of wild-type human FGF-1 protein (SEQ ID NO: 4),
wherein the numbering of the amino acid positions is based on the numbering scheme for the 140 amino acid form of human FGF-1, and
wherein the composition is administered without heparin.

22. The method of claim 21, wherein the mutant FGF protein binds specifically to at least one FGF receptor (FGFR) and triggers growth or proliferation of cultured fibroblast cells.

23. The method of claim 21, wherein the mutant FGF protein has a greater functional half-life than wild-type human FGF-1 according to a cultured fibroblast proliferation assay.

24. The method of claim 21, wherein the protein structure of the mutant FGF protein has a greater thermodynamic stability than wild-type human FGF-1.

25. The method of claim 24, wherein the mutant FGF protein has a ΔG_{unfolding} value greater than that of wild-type human FGF-1 such that
ΔΔG=ΔG_{unfolding}wild-type−ΔG_{unfolding}mutant<0
according to one or more of the following methods: isothermal equilibrium denaturation, differential scanning calorimetry, or temperature-dependent spectroscopy.

26. The method of claim 21, wherein the ischemic condition or disease is coronary artery disease or peripheral vascular disease.

27. The method of claim 21, wherein the composition is administered locally at or near a site of the ischemic condition or disease within a body of the individual.

28. The method of claim 21, wherein the composition is administered locally at or near a site within a body of the individual causing the ischemic condition or disease.

29. The method of claim 28, wherein the composition is administered locally at or near an occluded blood vessel.


SEQUENCE LISTING (4 sequences)

SEQ ID NO: 1 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 140 aa, Artificial Sequence
  Mutant human FGF-1 protein
FNLPPGNYKKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 3 - protein, 140 aa, Artificial Sequence
  Mutant human FGF-1 protein
FNLPPGNYKKPKLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 4 - protein, 134 aa, Artificial Sequence
  Mutant human FGF-1 protein
FNLPPGNYKKPVLLYCSNGGHFLRILPDGTVDGTRDRSDQHIQFQLSAESVGEVYIKSTETGQYLAIDTDGLVYGSQTPNEETLFLERLEENHYNTYISKKHGWFLGIKKNGSVKGTHYGQKAILFLPLPVSSD